(19) United States
(12) Patent Application Publication
(10) Pub. No.: US 2020/0140786 A1
(43) Pub. Date: May 7, 2020

(54) USE OF ENZYME FOR WASHING, METHOD FOR WASHING AND WAREWASHING COMPOSITION

(71) Applicant: NOVOZYMES A/S, Bagsvaerd (DK)

(72) Inventors: Rasmus Rune Hansen, Ballerup (DK); Rasmus Boedker Lassen, Bagsvaerd (DK)

(73) Assignee: NOVOZYMES A/S, Bagsvaerd (DK)

(21) Appl. No.: 16/338,041

(22) PCT Filed: Sep. 27, 2017
(86) PCT No.: PCT/EP2017/074432

§ 371 Date: Mar. 29, 2019

(30) Foreign Application Priority Data
Sep. 29, 2016 (EP) 16191330.6
Dec. 7, 2016 (EP) 16202644.7
Apr. 21, 2017 (EP) 17167438.5

(51) Int. Cl. C11D 3/386; C12N 9/26; C12N 9/50; C11D 11/00

(52) U.S. Cl. CPC C11D 3/38618; C12N 9/2414; C11D 11/0023; C11D 3/3869; C12N 9/50

(57) ABSTRACT

The present invention concerns the use of an amylase and/or a protease for removing and/or reducing soil on a surface, a method for removing and/or reducing soil on a surface and a warewashing composition.

### FIELD OF THE INVENTION

The present invention concerns the use of an amylase and/or a protease for removing and/or reducing soil on a surface, a method for removing and/or reducing soil on a surface and a warewashing composition.

### REFERENCE TO A SEQUENCE LISTING

This application comprises a Sequence Listing in computer readable form, which is incorporated herein by reference.

### Overview of Sequences Listing

SEQ ID NO: 1 is the amino acid sequence of an alpha-amylase (AA560)
SEQ ID NO: 2 is the amino acid sequence of an alpha-amylase (SP722)
SEQ ID NO: 3 is the amino acid sequence of an alpha-amylase (Termamyl)
SEQ ID NO: 4 is the amino acid sequence of a fusion alpha-amylase
SEQ ID NO: 5 is the amino acid sequence of a protease (Bacillus Clausii)
SEQ ID NO: 6 is the amino acid sequence of a protease (Bacillus licheniformis)
SEQ ID NO: 7 is the amino acid sequence of a protease (Bacillus halodurans)
SEQ ID NO: 8 is the amino acid sequence of a protease (Bacillus amyloliquefaciens, BNP′)
SEQ ID NO: 9-17 are the amino acid sequence of a subtilisin inhibitors.
SEQ ID NO: 18 is the amino acid sequence of a licheninase (Bacillus agaradhaerens)
SEQ ID NO: 19 is the amino acid sequence of a licheninase (Bacillus sp-62449)
SEQ ID NO: 20 is the amino acid sequence of a licheninase (Bacillus sp-62449)
SEQ ID NO: 21 is the amino acid sequence of a licheninase (Bacillus akibai)
SEQ ID NO: 22 is the amino acid sequence of a licheninase (Bacillus mojavensis)
SEQ ID NO: 23 is His-tagged recombinant mature amino acid sequence of the licheninase of SEQ ID NO: 18
SEQ ID NO: 24 is the amino acid sequence of a variant protease.

### FIGURES

FIG. 1 shows wash performance of a mild pH warewashing detergent composition with and without amylase and/or protease.

FIG. 2 shows a wash performance of a mild pH warewashing detergent composition and commercial available warewashing detergent compositions.

### BACKGROUND OF THE INVENTION

Industrial and institutional warewashing is a warewashing process applied in an industrial, commercial or institutional situation to provide clean and hygienic ware in as short a timeframe as possible. To achieve this result, ware washers generally apply high temperatures and strong mechanical and strong chemical action during the washing process.

In the majority of ware wash applications, the time available for washing is limited due to capacity constraints. Generally, a wash cycle is between 10 seconds and 5 minutes. In order to overcome these time constraints and deliver clean and hygienic ware, ware washers generally apply a high level of mechanical action to the ware. This is generally done using high pressure water distributed through nozzles and that is re-circulated in the ware washer. In some cases, an abrasive element can be introduced to the system (for example polymer beads) to enhance the mechanical effect of the water on the soiled ware. Despite the high degree of mechanical action applied in warewashing processes, a strong chemical action is relied upon to deliver the required levels of cleanliness and if required, hygiene. Ware wash chemicals are characterized by generally being highly alkaline and containing other elements to enhance the cleaning performance to ensure a satisfactory result, and to protect the ware wash machine from the potentially corrosive alkaline chemicals.

Warewashing detergent compositions for industrial and institutional warewashing comprise harsh chemical designed to remove the stains and soil from the ware. As an example the industrial warewashing compositions comprises up to 50% sodium hydroxide resulting in the pH of the composition being above 10.5 and with a pH up to 13.5. The high pH detergents pose a safety concern because many operators are not properly trained. The operators working with the warewashing detergents are thus exposed to corrosive chemicals which mean additional precautions when they operate the ware washer. Further the harsh chemical in the compositions pose a problem surrounding and the environment.

### SUMMARY OF THE INVENTION

The present invention concerns the use of an amylase and/or a protease for removing and/or reducing soil on a surface, wherein the surface is exposed to the amylase and/or protease for a time period of 10 to 240 seconds.

Further is claimed a method for removing and/or reducing soil on a surface, wherein a cleaning cycle comprises the steps of:
i. A washing step, wherein the surface is exposed to a wash liquor comprising
   a. an amylase and/or a protease and optionally detergent components, or
   b. a warewashing detergent composition according to the invention,
ii. Optionally draining part of the wash liquor,
iii. Optionally rinsing the surface,
iv. Optionally drying the surface;

wherein the surface is exposed to the wash liquor for a time period in the range of 10 to 240 seconds.

The invention also concerns a warewashing detergent composition comprising an amylase and/or a protease and one or more detergent components which composition has a pH in the range of 7-10.5.

### Definitions

Clean water: By the term “clean water” is meant water that has not been used as wash liquor in a previous cleaning cycle.

Cleaning cycle: The term “cleaning cycle” is defined herein as a cleaning operation wherein a ware is contacted to a wash liquor for a period of time by circulating the wash liquor and spraying the wash liquor onto the ware in order to clean the ware. The ware is optionally rinsed and dried.

Detergent component: The term “detergent component” refers to the ingredients that can be comprised in a warewashing detergent composition. Such components that can facilitate the cleaning process. Examples of detergent components are surfactants, builders, chelators or chelating agents, bleach system or bleach components, polymers, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

Warewashing detergent composition: The term “warewashing detergent composition” refers to compositions that find use in the removal and/or reduction of undesired compounds from surfaces to be cleaned, such as surfaces of ware or the surfaces present in the interior of a warewashing machine.

The warewashing detergent composition can be any compositions intended for reducing and/or removing soil from dishes, table ware, pots, pans, cutlery and all forms of compositions for reducing and/or removing soil from the inner surfaces in dishwashing machines. The present invention is not restricted to any particular type of warewashing detergent composition. The terms encompass any detergent component selected for the particular type of cleaning composition desired and the form of the product (e.g., liquid, gel, powder, bar, granulate, paste, or spray compositions). The detergent composition can contain enzymes in addition to the amylase and/or a protease comprised in the composition, e.g. one or more additional enzymes such as proteases, amylases, lipases, cutinases, cellulases, endoglucanases, xyloglucanases, pectinases, pectin lyases, xanthanases, peroxidaes, haloperoxygenases, catalases and mannanases, or any mixture thereof and a detergent component.

Warewashing machine: The term “warewashing machine” means any kind of washing machine that can be used for industrial or institutional warewashing. The term includes but are not limited to a door warewashing machine, a hood warewashing machine, a conveyor warewashing machine, an undercounter warewashing machine, a glasswasher, a flight warewashing machine, a pot and pan warewashing machine and a utensil washer.

Ware: The term “ware” is intended to mean any form of dishes, kitchen utensil, dinner set or tableware such as but not limited to pans, plates, drinking glasses, cups, knives, forks, spoons, porcelain etc. The ware can be made of any suitable material such as metal, glass, rubber, plastic, PVC, acrylics, ceramics, china or porcelain.

Improved wash performance: The term “improved wash performance” is defined herein as an enzyme or blend of enzymes displaying an increased wash performance relative to the wash performance of a similar wash without the enzyme or without the blend of enzymes e.g. by increased soil removal or increased soil reduction.

Wash performance: The term “wash performance” is used as an enzyme's ability to remove or reduced soil present on a surface to be cleaned during e.g. a cleaning cycle.

Variant: The term “variant” means a polypeptide having same activity as the parent enzyme comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding an amino acid adjacent to and immediately following the amino acid occupying a position.

Wash liquor: The term “wash liquor” is intended to mean the solution or mixture of water and detergents optionally including enzymes used for hard surface cleaning or for dishwashing.

The term “water hardness” or “degree of hardness” or “dH” or “° dH” as used herein refers to German degrees of hardness. One degree is defined as 10 milligrams of calcium oxide per litre of water.

### Conventions for Designation of Variants

For purposes of the present invention, the polypeptides disclosed in SEQ ID NO: 1, 2, 3, 4, 5, 6, 7, 8, 9, 10, 11, 12, 13, 14, 15, 16, 17, 18, 19, or 20 may be used to determine the corresponding amino acid residue in another polypeptide. The amino acid sequence of another polypeptide is aligned with the polypeptide disclosed in SEQ ID NO: 1, 2, 3, 4, 5, 6, 7, 8, 9, 10, 11, 12, 13, 14, 15, 16, 17, 18, 19, or 20 depending on whether it is an alpha-amylase, a protease, a subtilisin inhibitor or a licheninase, and based on the alignment, the amino acid position number corresponding to any amino acid residue in the polypeptide disclosed in SEQ ID NO: 1, 2, 3, 4, 5, 6, 7, 8, 9, 10, 11, 12, 13, 14, 15, 16, 17, 18, 19, or 20 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

Identification of the corresponding amino acid residue in another enzyme may be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the polypeptide of SEQ ID NO: 1, 2, 3, 4, 5, 6, 7, 8, 9, 10, 11, 12, 13, 14, 15, 16, 17, 18, 19, or 20 such that traditional sequence-based comparison fails to detect their relationship (Lindahl and Elofsson, 2000, J. Mol. Biol. 295: 613-615), other pairwise sequence comparison algorithms may be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402). Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP super families of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

It is within the knowledge of the skilled person to determine which alignment tool to use when corresponding amino acid positions must be identified. Therefore, it is contemplated that any available alignment tool that the skilled person find suitable may be used in the context of the present invention.

In describing the enzyme variants described herein, the nomenclature described below is adapted for ease of reference. The accepted IUPAC single letter or three letters amino acid abbreviations are employed. Amino acid positions are indicated with H1, G109, etc.

Variants described herein comprises one or more modifications as compared to the parent polypeptide. Accordingly, variants may comprise conservative modifications, in particular, such conservative modifications may be conservative substitutions. Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Asn/Gln, Thr/Ser, Ala/Gly, Ala/Thr, Ser/Asn, Ala/Val, Ser/Gly, Tyr/Phe, Ala/Pro, Lys/Arg, Asp/Asn, Glu/Gln, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Substitutions:

For an amino acid substitution, the following nomenclature is used: Original amino acid, position, substituted amino acid. Accordingly, the substitution of glycine at position G109 with alanine is designated as “Gly109Ala” or “G109A”. Multiple mutations are separated by addition marks (“+”) or by commas (“,”), e.g., “Gly109Ala+Leu173Pro” or “G109A,L173P”, representing substitutions at positions 109 and 173 of glycine (G) with alanine (A) and leucine (L) with proline (P), respectively. If more than one amino acid may be substituted in a given position these are listed or divided by slash, such as /. Thus, if both Ala and Pro according to the invention may be substituted instead of the amino acid occupying at position 109 this is indicated as X109A/P where the X in the present example indicates that different enzymes may be parent e.g. such as an alpha-amylase with SEQ ID NO: 1 or an alpha-amylase having at least 75% identity hereto. Thus, in some cases the variants are represented as 109A/P or X109A/P indicating that the amino acids to be substituted vary depending on the parent enzyme.

Deletions:

For an amino acid deletion, the following nomenclature is used: Original amino acid, position, *. Accordingly, the deletion of arginine at position 181 is designated as “Arg181*” or “R181*”. Multiple deletions are separated by addition marks (“+”) or commas, e.g., “Arg181*+Gly182*” or “R181*+G182*” or “R181*, G182*”.

Insertions:

The insertion of an additional amino acid residue such as e.g. a lysine after G #₁ may be indicated by: Gly #₁GlyLys or G #₁GK. Alternatively insertion of an additional amino acid residue such as lysine after G109 may be indicated by: *109aL. When more than one amino acid residue is inserted, such as e.g. a Lys, and Ala after 109 this may be indicated as: Gly109GlyLysAla or G109GKA. In such cases, the inserted amino acid residue(s) may also be numbered by the addition of lower case letters to the position number of the amino acid residue preceding the inserted amino acid residue(s), in this example: *109aK*109bA.

Collectively, substitutions, deletions, and insertions may herein be termed “modifications”. Thus, it is to be understood that any variant described herein comprises modifications, such as substitutions, deletions and/or insertions unless otherwise indicated by context.

Multiple Modifications:

Variants comprising multiple modifications are separated by addition marks (“+”), slash marks (“/”), or by commas (“,”), e.g., “Gly109Pro+Lys391Ala” or “G109P, K391A” representing a substitution of glycine at position 109 and lysine at position 391 with proline and alanine, respectively as described above.

Different Modifications:

Where different modifications can be introduced at a position, the different modifications are separated by a division (“/”), or by a comma (“,”), e.g., “Gly109Pro,Lys” or “G109P,K” represents a substitution of glycine at position 109 with proline or lysine. Thus, “Gly109Pro,Lys+Lys391Ala” designates the following variants: “Gly109Pro+Lys391Ala”, “Gly109Lys+Lys391Ala” or “G109P,K+K391A”.

### DETAILED DESCRIPTION OF THE INVENTION

Industrial and institutional warewashing is a process applied in an industrial, commercial or institutional situation to provide clean and hygienic ware in as short a timeframe as possible. To achieve this result, ware washers generally apply high temperatures and strong mechanical and strong chemical action in the washing process.

The inventors have found a more environmentally friendly way of carrying out warewashing without the use of harsh chemical. The new use and method relates to using amylase and/or protease in the warewashing process. When enzymes are used for removing and/or reducing soil from surfaces to be washed it is possible to lower the pH of the warewashing detergent compositions. Using less harsh chemicals has an impact on the environment and on the safety for the operator performing the wash process.

The inventors have found that use of an amylase and/or a protease for removing and/or reducing soil on a surface in a warewashing process is actually possible. It is of utmost importance that warewashing in industrial kitchens e.g. in a restaurant is carried out in very short time such as few minutes or even less than a minute. In one embodiment of the invention the surface is exposed to the amylase and/or protease for a time period of 10 to 240 seconds. In addition, the invention concerns a method for removing and/or reducing soil on a surface, wherein a cleaning cycle comprises the steps of:
i. A washing step, wherein the surface is exposed to a wash liquor comprising
   a. an amylase and/or a protease and optionally detergent components, or
   b. a warewashing detergent composition according to the invention,
ii. Optionally draining part of the wash liquor,
iii. Optionally rinsing the surface,
iv. Optionally drying the surface;

wherein the surface is exposed to the wash liquor for a time period in the range of 10 to 240 seconds.

The surface may be exposed to the amylase and/or protease for a time period of 10 to 220 seconds, such in the range of 10 to 200 seconds, in the range of 10 to 180 seconds, in the range of 10 to 160 seconds, in the range of 10 to 140 seconds, in the range of 10 to 120 seconds, in the range of 10 to 100 seconds, in the range of 10 to 80 seconds, in the range of 10 to 70 seconds, in the range of 10 to 66 seconds, in the range of 20 to 66 seconds, in the range of 25 to 66 seconds, in the range of 28 to 66 seconds, in the range of 28 to 60 seconds, in the range of 28 to 55 seconds, in the range of 28 to 50 seconds or in the range of 28 to 45 seconds.

The inventor has demonstrated that the amylase or protease used separately shows improved wash performance and when using the enzymes together, the enzymes exhibit synergistic effect.

Another advantage of using amylase and/or protease in the inventive method is that the enzymes prevent redeposition of soil on the surface. In one embodiment a protease is used in the inventive method and prevents redeposition of soil on a surface to be cleaned. In another embodiment of the invention an amylase is used in the inventive method and prevents redeposition of soil on a surface to be cleaned. In a third embodiment of the invention a protease is used together with an amylase in the inventive method and prevents redeposition of soil on a surface to be cleaned.

In one embodiment of the invention the cleaning cycle comprises a rinsing step and/or a drying step. In one embodiment clean water is used for the rinsing step optionally together with a rinse aid. The rinse aid can comprise sodium xylenesulfonate, isopropyl alcohol, amine polyglycol condensate, alcohol alkoxylate, hydroxyacetic acid, alcohol alkoxylate, polyoxypropylene-polyoxyethylene, citric acid, urea, acrylic acid, alkoxylated alcohol, 2-propenoic acid telemer, sodium salt, triarylmethane and methyl-oxirane polymer with oxirane.

In one embodiment the method comprises a soaking step before step a.

During operation of ware wash processes, the inner surfaces of the warewash machine are exposed to water containing potentially high levels of organic soils, and over time a soil film or deposit can form on the inner surfaces of the ware wash machine. This film can potentially be resistant to removal during normal daily cleaning operations. In ware wash processes where a soil film is present on the inner surfaces of the machine, it is not uncommon to see reduced performance, increase chemical dosing requirements, malodours and the formation of biofilms within the machine.

The cleaning cycle can comprise a draining step, wherein part of the wash liquor is drained. In one embodiment the clean water from rinsing step c replaces the wash liquor drained. 5-15% of the wash liquor can be replaced by clean water, such as 7.5% of the wash liquor is replaced by clean water. The wash liquor can be re-used in a subsequent cleaning cycle. The pH of the wash liquor may be in the range of 7.5-10.5, such as in the range of 7.5-10, in the range of 7.5-9.5, in the range of 7.5-9.0, in the range of 7.5-8.5, in the range of 7.5-8.2, or in the range of 7.8-8.2. The temperature of the wash liquor may be in the range of 50-95° C., such as in the range of 50-90° C., in the range of 50-85° C., in the range of 50-80° C., in the range of 50-75° C., in the range of 50-70° C., in the range of 50-65° C., 55-62° C. or in the range of 58-62° C.

The surface may undergo the cleaning cycle 1 time, 2 times, 3 times, 4 times or even 5 times.

In one embodiment of the invention the wash liquor comprises one or more detergent components selected from the group consisting of surfactants, builders, chelators or chelating agents, bleach system or bleach components, polymers, foam boosters, suds suppressors, dyes, perfume, tannish inhibitors, optical brighteners, bactericides, fungicides, soil suspending agents, anti-corrosion agents, enzyme stabilizers, enzyme inhibitors or activators, transferase(s), hydrolytic enzymes, oxido reductases, bluing agents and fluorescent dyes, antioxidants, and solubilizers. The detergent components can be added separately to the wash liquor or as a warewashing detergent composition comprising detergent components and an amylase and/or a protease. The warewashing composition according to the invention comprises an amylase and/or a protease and one or more detergent components which composition has a pH in the range of 7-10.5. The composition may comprise at least 5% wt. of a chelator and/or less than 2% of a surfactant. In one embodiment of the invention, the composition does not comprise a surfactant. The composition is a warewashing composition for industrial or institutional use and may have a pH in the range of 7.5-10.5, such as in the range of 7.5-10, in the range of 7.5-9.5, in the range of 7.5-9.0, in the range of 7.5-8.5, in the range of 7.5-8.2, or in the range of 7.8-8.2. The composition is thus lower in pH than other warewashing detergent composition and can therefore be used without safety concerns for the operator or for the environment.

The concentration of the warewashing detergent composition is in the range of 0.5-5 g/liter wash liquor, such as in the range of 1-4 g/liter wash liquor, such as in the range of 1.5-3 g/liter wash liquor. In a preferred embodiment the concentration is 2 g/liter wash liquor.

Given the broad range of potential applications of warewashers, there is a large variety of systems available. These include single wash undercounter systems (similar to household dishwashers), hooded single use systems, systems for larger or heavily soiled equipment and large conveyor or flight machines that operate continuously. Warewashers commonly contain a sump or reservoir of washing water. The purpose of this sump is to reduce water and ware wash chemical consumption by allowing the re-use and re-circulation of the water over a period of time or washes. This saves water and energy for heating the water.

The invention can be carried out in a variety of warewashing machines, including industrial warewashing machines.

The method for removing and/or reducing soil on a surface can be carried out in a warewashing machine selected from the group consisting of a door warewashing machine, a hood warewashing machine, a conveyor warewashing machine, an undercounter warewashing machine, a glasswasher, a flight warewashing machine, a pot and pan warewashing machine and a utensil washer. In one embodiment of the invention the surface to be cleaned is the inner surface of a warewashing machine or the surface of a ware.

The composition can be in the form of a bar, a block a homogenous tablet, a tablet having two or more layers, a pouch having one or more compartments, a regular or compact powder, a granule, a paste, a gel, or a regular, compact or concentrated liquid.

A solid warewashing detergent composition includes an effective amount of cleaning agent and an alkaline source to provide soil removal, solidification agent for binding the composition, and branched fatty acid disintegrator to provide improved dissolution of the solid detergent composition into aqueous use solution. The branched fatty acid disintegrator selected from the group consisting of sodium isononanoate, isononanoic acid, sodium isooctanoate, isooctanoic acid, sodium neodecanoate, neodecanoic acid, sodium neopentanoate, neopentanoic acid, sodium neoheptanoate, neoheptanoic acid, 3,5,5-trimethylhexanoic acid, 6-methyl-heptanoic acid, 2,2-dimethyloctanoic acid, neopentanoic acid (2,2-dimethylpropanoic acid), 2,2-dimethylpentanoic acid, and salts thereof, or mixtures thereof.

In one embodiment the composition is a block or a tablet having a dissolution rate when exposed to 4000 mL of aqueous solution at 68° C. of at least 15 g/minute.

In one embodiment the composition is a liquid warewashing composition.

The wash liquor and the warewashing composition can comprise an additional enzyme selected from the group consisting of an additional protease, lipase, cutinase, an additional amylase, carbohydrase, cellulase, pectinase, mannanase, arabinase, galactanase, xylanase, oxidase, a licheninase, a laccase and/or peroxidase. The wash liquor can be supplied with enzymes and the detergent components from separate containers and further the amylase and protease and optionally additional enzymes can be supplied from separate containers.

The warewashing composition may comprise one or more protease inhibitors or one or more protease inhibitors may be comprised in the wash liquor. At least one of the protease inhibitors is a peptide aldehyde, a hydrosulfite adduct or a hemiacetal adduct thereof. One or more protease inhibitor(s) is (are) typically added to improve the stability of the warewashing composition, in particular for liquid complsitions, and in this way improve the shelf life of the composition so is can be stored for a longer periode before use and still provide same performance in the warewashing process.

In one embodiment the protease inhibitor is a peptide aldehyde of the formula P-(A)y-L-(B)x-B0-H or a hydrosulfite adduct or hemiacetal adduct thereof, wherein:

i. H is hydrogen;

ii. B0 is a single amino acid residue with L- or D-configuration of the formula —NH—CH(R)—C(═O)—;

iii. x is 1, 2 or 3 for (B)x, and B is independently a single amino acid connected to B0 via the C-terminal of the (B)x amino acid

iv. L is absent or L is independently a linker group of the formula —C(═O)—, —C(═O)—C(═O)—, —C(═S)—, —C(═S)—C(═S)— or —C(═S)—C(═O)—;

v. y is 0, 1 or 2 for (A)y, and A is independently a single amino acid residue connected to L via the N-terminal of the (A)y amino acid, with the proviso that if L is absent then A is absent;

vi. P is selected from the group consisting of hydrogen and an N-terminal protection group, with the proviso that if L is absent then P is an N-terminal protection group;

vii. R is independently selected from the group consisting of C1-6 alkyl, C6-10 aryl or C7-10 arylalkyl optionally substituted with one or more, identical or different, substituent's R′;

viii. R′ is independently selected from the group consisting of halogen, —OH, —OR″, —SH, —SR″, —NH2, —NHR″, —NR″2, —CO2H, —CONH2, —CONHR″, —CONR″2, —NHC(═N)NH2; and

ix. R″ is a C1-6 alkyl group.

In another embodiment the protease inhibitor is a hydrosulfite adduct of a peptide aldehyde is of the formula P-(A)y-L-(B)x-N(H)—CHR—CH(OH)—SO3M, wherein

i. M is hydrogen or an alkali metal;

ii. x is 1, 2 or 3 for (B)x, and B is independently a single amino acid connected to B0 via the C-terminal of the (B)x amino acid

iii. L is absent or L is independently a linker group of the formula —C(═O)—, —C(═O)—C(═O)—, —C(═S)—, —C(═S)—C(═S)— or —C(═S)—C(═O)—;

iv. y is 0, 1 or 2 for (A)y, and A is independently a single amino acid residue connected to L via the N-terminal of the (A)y amino acid, with the proviso that if L is absent then A is absent;

v. P is selected from the group consisting of hydrogen and an N-terminal protection group, with the proviso that if L is absent then P is an N-terminal protection group;

vi. R is independently selected from the group consisting of C1-6 alkyl, C6-10 aryl or C7-10 arylalkyl optionally substituted with one or more, identical or different, substituent's R′;

vii. R′ is independently selected from the group consisting of halogen, —OH, —OR″, —SH, —SR″, —NH2, —NHR″, —NR″2, —CO2H, —CONH2, —CONHR″, —CONR″2, —NHC(═N)NH2; and

viii. R″ is a C1-6 alkyl group.

The peptide inhibitors are described in further details below.
The amylase used for removing and/or reducing soil on a surface can be an alpha-amylase. The alpha-amylase can be comprised in the wash liquor or in the warewashing detergent composition.
In one embodiment of the invention the amylase has a sequence identity of at least 70%, such as at least 75%, such as at least 80%, such as at least 85%, such as at least 90%, such as at least 95%, such as at least 98%, such as at least 99%, such as 100%, to the sequence of SEQ ID NO: 1, 2, 3 or 4.

In one embodiment of the invention a licheninase is used together with the amylase and/or the protease in a warewashing process. The licheninase can be the licheninase of SEQ ID NO. 18, SEQ ID NO. 19, SEQ ID NO. 20, SEQ ID NO. 21, SEQ ID NO. 22 or a His-tagged version of the enzyme. In one embodiment of the invention the licheninase is the enzyme of amino acid sequence NO. 23. Testing an enzyme for licheninase activity can be determined as described in example 1 of European patent application number 15198277.4. The licheninases can be cloned, expressed and purified as described in example 2 of same patent application.

In one embodiment the amylase is an alpha-amylase variant comprising a modification in one or more positions corresponding to positions D183*+G184*+R118K+N195F+R320K+R458K or M9L+R118K+G149A+G182T+G186A+D183*+G184*+N195F+M202L+T2571+Y295F+N299Y+R320K+M323T+A339S+E345R+R458K of SEQ ID NO: 1; D183*+G184* or W140Y+D183*+G184*+N195F+V206Y+Y243F+E260G+G304R+G476K of SEQ ID NO: 2; H156Y+A181T+N190F+A209V+Q264S of SEQ ID NO: 3, wherein said alpha-amylase variant has a sequence identity of at least 75% but less than 100% to SEQ ID NO: 1, 2 or 3, respectively, and wherein said alpha-amylase variant has alpha-amylase activity.

In one embodiment the amylase is an alpha-amylase variant comprising a modification in one or more positions corresponding to positions H1, N54, V56, K72, G109, F113, R116, T134, W140, W159, W167, Q169, Q172, L173, A174, R181, G182, D183, G184, W189, E194, N195, V206, G255, N260, F262, A265, W284, F289, S304, G305, W347,K391, Q395, W439, W469, R444, F473, G476, and G477 of SEQ ID NO: 4, wherein said alpha-amylase variant has a sequence identity of at least 75% but less than 100% to SEQ ID NO: 4 and wherein said alpha-amylase variant has alpha-amylase activity.

In one embodiment the said modification in one or more positions is selected from the group consisting of: H1*, H1A, N54S, V56T, K72R, G109A, F113Q, R116Q, R116H, T134E, W140Y, W140F, W140H, W159Y, W159F, W159H, W167Y, W167H, W167F, Q169E, Q172K, Q172G, Q172N, L173P, A174*, A174S, R181*, G182*, D183*, G184*, G184T, W189Y, W189F, W189H, W189E, W189D, W189Q, W189N, E194D, E194N, E194S, N195F, V206L, V206F, V206Y, G255A, N260G, N260P, N260A, N260G, N260P, N260A, A265G, W284G, W284H, F289H, S304K, S304R, S304Q, S304E, G305K, G305R, G305Q, G305E, W347Y, W347F, W347H, K391A, Q395P, W439N, W439Q, W439T, R444Q, W469T, W469N, F473R, G476R, G476Q, G476E, G476K G477K, G477R, G477Q, and G477E wherein the positions correspond to positions of SEQ ID NO: 4.

In one embodiment the alpha-amylase variant comprises a deletion in the positions corresponding to R181+G182; R181+D183; R181+G184; G182+D183; G182+G184; or D183+G184 of SEQ ID NO:4.

In one embodiment the alpha-amylase variant is selected from the group consisting of:

### H1*+N54S+V56T+G109A+Q169E+Q172K+A174*+G182*+D183*+N195F+V206L+K391A+G476K;

### H1*+N54S+V56T+G109A+R116H+A174S+G182*+D183*+N195F+V206L+K391A+G476K;

### H1*+N54S+V56T+K72R+G109A+F113Q+R116Q+W167F+Q172G+A174S+G182*+D183*+G184T+N195F+V206L+K391A+P473R+G476K;

### H1*+N54S+V56T+G109A+F113Q+R116Q+Q172N+A174S+G182*+D183*+N195F+V206L+A265G+K391A+P473R+G476K;

### H1*+N54S+V56T+K72R+G109A+F113Q+W167F+Q172R+A174S+G182*+D183*+N195F+V206L+K391A+G476K;

### H1*+N54S+V56T+K72R+G109A+R116H+T134E+W167F+Q172G+L173V+A174S+G182*+D183*+N195F+V206L+G255A+K391A+G476K;

### H1*+N54S+V56T+K72R+G109A+R116H+T134E+W167F+Q172G+L173V+A174S+G182*+D183*+N195F+V206L+G255A+K391A+Q395P+T444Q+P473R+G476K;

### H1*+N54S+V56T+G109A+T134E+A174S+G182*+D183*+N195F+V206L+K391A+G476K;

### H1*+N54S+V56T+K72R+G109A+A174S+G182*+D183*+N195F+V206L+G255A+K391A+G476K;

H1*+N54S+V56T+K72R+G109A+F113Q+R116Q+W167F+Q172G+A174S+G184T+N195F+V206L+K391A+P473R+G476K, and H1*+N54S+V56T+G109A+W167F+Q172E+L173P+A174K+G182*+D183*+N195F+V206L+K391A+G476K, of the polypeptide of SEQ ID NO: 4 and wherein said alpha-amylase variant shares at least 80%, such as at least 85%, such as at least 90%, such as at least 93%, such as at least 94%, such as at least 95%, such as at least 96%, such as at least 97%, such as at least 98%, but less than 100% sequence identity with the polypeptide of SEQ ID NO: 4 and wherein said alpha-amylase variant has alpha-amylase activity.

In one embodiment the protease is selected from the group consisting of

i. a protease having a sequence identity of at least 70%, such as at least 75%, such as at least 80%, such as at least 85%, such as at least 90%, such as at least 95%, such as at least 98%, such as at least 99%, such as 100%, to the sequences of SEQ ID NOs: 5, 6, 7 and 8;

ii. a protease variant comprising a substitution at one or more positions corresponding to positions 9, 15, 36, 61, 68, 76, 99, 106, 120, 167, 170, 194, 195, 205, 218, 235, 245 or 261 of SEQ ID NO: 5, wherein said protease variant has a sequence identity of at least 75% but less than 100% to SEQ ID NO: 5.

Wherever protease variant are described the positions refer to BPN′ numbering (SEQ ID NO: 8).

In one embodiment the protease is selected from the group consisting of: M222S, *36D+N76D+N120D+G195E+K235L, Y167A+R170S+A194P, S99SE, V68A+S106A, S9R+A15T+V68A+N218D+Q245R, S9R+A15T+G61E+V68A+A194P+V205I+Q245R+N261D and S99AD of SEQ ID NO: 5, wherein said protease variant has a sequence identity of at least 75% but less than 100% to SEQ ID NO: 5 and wherein said protease variant has protease activity. In one embodiment of the invention the protease is the protease variant of SEQ ID NO: 24.

Protease Inhibitor

The protease inhibitor maybe any compound which stabilises or inhibits the protease so that the protease or other enzyme(s) in the laundry soap bar are not degraded. Examples of protease inhibitors are aprotinin, bestatin, calpain inhibitor I and II, chymostatin, leupeptin, pepstatin, phenylmethanesulfonyl fluoride (PMSF), boric acid, borate, borax, boronic acids, phenylboronic acids such as 4-formylphenylboronic acid (4-FPBA), peptide aldehydes or hydrosulfite adducts or hemiacetal adducts thereof and peptide triflouromethyl ketones. There may be one or more protease inhibitors, such as 5, 4, 3, 2 or 1 inhibitor(s) of which at least one is a peptide aldehyde, a hydrosulfite adduct or a hemiacetal adduct thereof.

Peptide Aldehyde Inhibitor

The peptide aldehyde may have the formula P-(A)_{y}-L-(B)ₓ—B⁰—H or a hydrosulfite adduct or hemiacetal adduct thereof, wherein:
i. H is hydrogen;
ii. B⁰ is a single amino acid residue with L- or D-configuration of the formula —NH—CH(R)—C(═O)—;
iii. x is 1, 2 or 3 for (B)ₓ, and B is independently a single amino acid connected to B⁰ via the C-terminal of the B amino acid
iv. L is absent or L is independently a linker group of the formula —C(═O)—, —C(═O)—C(═O)—, —C(═S)—, —C(═S)—C(═S)— or —C(═S)—C(═O)—;
v. y is 0, 1 or 2 for (A)_{y}, and A is independently a single amino acid residue connected to L via the N-terminal of the A amino acid, with the proviso that if L is absent then A is absent;
vi. P is selected from the group consisting of hydrogen and an N-terminal protection group, with the proviso that if L is absent then P is an N-terminal protection group;
vii. R is independently selected from the group consisting of C₁₋₆ alkyl, C₆₋₁₀ aryl or C₇₋₁₀ arylalkyl optionally substituted with one or more, identical or different, substituent's R′;
viii. R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂; and
ix. R″ is a C₁₋₆ alkyl group.

x may be 1, 2 or 3 and therefore B may be 1, 2 or 3 amino acid residues respectively. Thus, B may represent B¹, B²—B¹ or B³—B²—B¹, where B³, B² and B¹ each represent one amino acid residue. y may be 0, 1 or 2 and therefore A may be absent, or 1 or 2 amino acid residues respectively having the formula A¹ or A²-A¹ wherein A² and A¹ each represent one amino acid residue.

B⁰ may be a single amino acid residue with L- or D-configuration, which is connected to H via the C-terminal of the amino acid, wherein R is a C₁₋₆ alkyl, C₆₋₁₀ aryl or C₇₋₁₀ arylalkyl side chain, such as methyl, ethyl, propyl, isopropyl, butyl, isobutyl, phenyl or benzyl, and wherein R may be optionally substituted with one or more, identical or different, substituent's R′. Particular examples are the D- or L-form of arginine (Arg), 3,4-dihydroxyphenylalanine, isoleucine (Ile), leucine (Leu), methionine (Met), norleucine (Nle), norvaline (Nva), phenylalanine (Phe), m-tyrosine, p-tyrosine (Tyr) and valine (Val). A particular embodiment is when B⁰ is leucine, methionine, phenylalanine, p-tyrosine and valine.

B¹, which is connected to B⁰ via the C-terminal of the B¹ amino acid, may be an aliphatic, hydrophobic and/or neutral amino acid. Examples of B¹ are alanine (Ala), cysteine (Cys), glycine (Gly), isoleucine (Ile), leucine (Leu), norleucine (Nle), norvaline (Nva), proline (Pro), serine (Ser), threonine (Thr) and valine (Val). Particular examples of B¹ are alanine, glycine, isoleucine, leucine and valine. A particular embodiment is when B¹ is alanine, glycine or valine.

If present, B², which is connected to B¹ via the C-terminal of the B² amino acid, may be an aliphatic, hydrophobic, neutral and/or polar amino acid. Examples of B² are alanine (Ala), arginine (Arg), capreomycidine (Cpd), cysteine (Cys), glycine (Gly), isoleucine (Ile), leucine (Leu), norleucine (Nle), norvaline (Nva), phenylalanine (Phe), proline (Pro), serine (Ser), threonine (Thr), and valine (Val). Particular examples of B² are alanine, arginine, capreomycidine, glycine, isoleucine, leucine, phenylalanine and valine. A particular embodiment is when B² is arginine, glycine, leucine, phenylalanine or valine.

B³, which if present is connected to B² via the C-terminal of the B³ amino acid, may be a large, aliphatic, aromatic, hydrophobic and/or neutral amino acid. Examples of B³ are isoleucine (lie), leucine (Leu), norleucine (Nle), norvaline (Nva), phenylalanine (Phe), phenylglycine, tyrosine (Tyr), tryptophan (Trp) and valine (Val). Particular examples of B³ are leucine, phenylalanine, tyrosine and tryptophan.

The linker group L may be absent or selected from the group consisting of —C(═O)—, —C(═O)—C(═O)—, —C(═S)—, —C(═S)—C(═S)— or —C(═S)—C(═O)—. Particular embodiments of the invention are when L is absent or L is a carbonyl group —C(═O)—.

A¹, which if present is connected to L via the N-terminal of the amino acid, may be an aliphatic, aromatic, hydrophobic, neutral and/or polar amino acid. Examples of A¹ are alanine (Ala), arginine (Arg), capreomycidine (Cpd), glycine (Gly), isoleucine (Ile), leucine (Leu), norleucine (Nle), norvaline (Nva), phenylalanine (Phe), threonine (Thr), tyrosine (Tyr), tryptophan (Trp) and valine (Val). Particular examples of A¹ are alanine, arginine, glycine, leucine, phenylalanine, tyrosine, tryptophan and valine. A particular embodiment is when B² is leucine, phenylalanine, tyrosine or tryptophan.

The A² residue, which if present is connected to A¹ via the N-terminal of the amino acid, may be a large, aliphatic, aromatic, hydrophobic and/or neutral amino acid. Examples of A² are arginine (Arg), isoleucine (Ile), leucine (Leu), norleucine (Nle), norvaline (Nva), phenylalanine (Phe), phenylglycine, Tyrosine (Tyr), tryptophan (Trp) and valine (Val). Particular examples of A² are phenylalanine and tyrosine.

The N-terminal protection group P (if present) may be selected from formyl, acetyl (Ac), benzoyl (Bz), trifluoroacetyl, methoxysuccinyl, aromatic and aliphatic urethane protecting groups such as fluorenylmethyloxycarbonyl (Fmoc), methoxycarbonyl, (fluoromethoxy)carbonyl, benzyloxycarbonyl (Cbz), t-butyloxycarbonyl (Boc) and adamantyloxycarbonyl; p-methoxybenzyl carbonyl (Moz), benzyl (Bn), p-methoxybenzyl (PMB), p-methoxyphenyl (PMP), methoxyacetyl, methylamino carbonyl, methylsulfonyl, ethylsulfonyl, benzylsulfonyl, methylphosphoramidyl (MeOP(OH)(═O)) and benzylphosphoramidyl (PhCH₂OP(OH)(═O)).

The general formula of the peptide aldehyde may also be written: P-A²-A¹-L-B³—B²B¹-B⁰—H, where P, A², A¹,L, B³, B², B¹ and B⁰ are as defined above.

In the case of a tripeptide aldehyde with a protection group (i.e. x=2, L is absent and A is absent), P is preferably acetyl, methoxycarbonyl, benzyloxycarbonyl, methylamino carbonyl, methylsulfonyl, benzylsulfonyl and benzylphosphoramidyl. In the case of a tetrapeptide aldehyde with a protection group (i.e. x=3, L is absent and A is absent), P is preferably acetyl, methoxycarbonyl, methylsulfonyl, ethylsulfonyl and methylphosphoramidyl.

Suitable peptide aldehydes are described in WO94/04651, WO95/25791, WO98/13458, WO98/13459, WO98/13460, WO98/13461, WO98/13462, WO07/141736, WO07/145963, WO09/118375, WO10/055052 and WO11/036153.

More particularly, the peptide aldehyde may be

Cbz-Arg-Ala-Tyr-H (L-Alaninamide, N2-[(phenylmethoxy)carbonyl]-L-arginyl-N-[(1S)-1-formyl-2-(4-hydroxyphenyl)ethyl]-),

Ac-Gly-Ala-Tyr-H (L-Alaninamide, N-acetylglycyl-N-[(1S)-1-formyl-2-(4-hydroxyphenyl)ethyl]-)

Cbz-Gly-Ala-Tyr-H (L-Alaninamide, N-[(phenylmethoxy)carbonyl]glycyl-N-[(1S)-1-formyl-2-(4-hydroxyphenyl)ethyl]-),

Cbz-Gly-Ala-Leu-H (L-Alaninamide, N-[(phenylmethoxy)carbonyl]glycyl-N-[(1S)-1-formyl-3-methylbutyl]-),

Cbz-Val-Ala-Leu-H (L-Alaninamide, N-[(phenylmethoxy)carbonyl]-L-valyl-N-[(1S)-1-formyl-3-methylbutyl]-),

Cbz-Gly-Ala-Phe-H (L-Alaninamide, N-[(phenylmethoxy)carbonyl]glycyl-N-[(1S)-1-formyl-2-phenylethyl]-),

Cbz-Gly-Ala-Val-H (L-Alaninamide, N-[(phenylmethoxy)carbonyl]glycyl-N-[(1S)-1-formyl-2-methylpropyl]-),

Cbz-Gly-Gly-Tyr-H (Glycinamide, N-[(phenylmethoxy)carbonyl]glycyl-N-[(1S)-1-formyl-2-(4-hydroxyphenyl)ethyl]-),

Cbz-Gly-Gly-Phe-H (Glycinamide, N-[(phenylmethoxy)carbonyl]glycyl-N-[(1S)-1-formyl-2-phenylethyl]-),

Cbz-Arg-Val-Tyr-H (L-Valinamide, N2-[(phenylmethoxy)carbonyl]-L-arginyl-N-[(1S)-1-formyl-2-(4-hydroxyphenyl)ethyl]-),

Cbz-Leu-Val-Tyr-H (L-Valinamide, N-[(phenylmethoxy)carbonyl]-L-leucyl-N-[(1S)-1-formyl-2-(4-hydroxyphenyl)ethyl]-)

Ac-Leu-Gly-Ala-Tyr-H (L-Alaninamide, N-acetyl-L-leucylglycyl-N-[(1S)-1-formyl-2-(4-hydroxyphenyl)ethyl]-),

Ac-Phe-Gly-Ala-Tyr-H (L-Alaninamide, N-acetyl-L-phenylalanylglycyl-N-[(1S)-1-formyl-2-(4-hydroxyphenyl)ethyl]-),

Ac-Tyr-Gly-Ala-Tyr-H (L-Alaninamide, N-acetyl-L-tyrosylglycyl-N-[(1S)-1-formyl-2-(4-hydroxyphenyl)ethyl]-),

Ac-Phe-Gly-Ala-Leu-H (L-Alaninamide, N-acetyl-L-phenylalanylglycyl-N-[(1S)-1-formyl-3-methylbutyl]-),

Ac-Phe-Gly-Ala-Phe-H (L-Alaninamide, N-acetyl-L-phenylalanylglycyl-N-[(1S)-1-formyl-2-phenylethyl]-)

Ac-Phe-Gly-Val-Tyr-H (L-Valinamide, N-acetyl-L-phenylalanylglycyl-N-[(1S)-1-formyl-2-(4-hydroxyphenyl)ethyl]-),

Ac-Phe-Gly-Ala-Met-H (L-Alaninamide, N-acetyl-L-phenylalanylglycyl-N-[(1S)-1-formyl-3-(methylthio)propyl]-),

Ac-Trp-Leu-Val-Tyr-H (L-Valinamide, N-acetyl-L-tryptophyl-L-leucyl-N-[(1S)-1-formyl-2-(4-hydroxyphenyl)ethyl]-),

MeO—CO-Val-Ala-Leu-H (L-Alaninamide, N-(methoxycarbonyl)-L-valyl-N-[(1S)-1-formyl-3-methylbutyl]-)

MeNHCO-Val-Ala-Leu-H (L-Alaninamide, N-(aminomethylcarbonyl)-L-valyl-N-[(1S)-1-formyl-3-methylbutyl]-),

MeO—CO-Phe-Gly-Ala-Leu-H (L-Alaninamide, N-(methoxycarbonyl)-L-phenylalanylglycyl-N-[(1S)-1-formyl-3-methyl butyl]-),

MeO—CO-Phe-Gly-Ala-Phe-H (L-Alaninamide, N-(methoxycarbonyl)-L-phenylalanylglycyl-N-[(1S)-1-formyl-2-phenylethyl]-),

MeSO2-Phe-Gly-Ala-Leu-H (L-Alaninamide, N-(methylsulfonyl)-L-phenylalanylglycyl-N-[(1S)-1-formyl-3-methylbutyl]-),

MeSO2-Val-Ala-Leu-H (L-Alaninamide, N-(methylsulfonyl)-L-valyl-N-[(1S)-1-formyl-3-methylbutyl]-),

PhCH2O—P(OH)(O)-Val-Ala-Leu-H (L-Alaninamide, N-[hydroxy(phenylmethoxy)phosphinyl]-L-valyl-N-[(1S)-1-formyl-3-methylbutyl]-),

EtSO2-Phe-Gly-Ala-Leu-H (L-Alaninamide, N-(ethylsulfonyl)-L-phenylalanylglycyl-N-[(1S)-1-formyl-3-methylbutyl]-),

PhCH2SO2-Val-Ala-Leu-H (L-Alaninamide, N-[(phenylmethyl)sulfonyl]-L-valyl-N-[(1S)-1-formyl-3-methylbutyl]-),

PhCH2O—P(OH)(O)-Leu-Ala-Leu-H (L-Alaninamide, N-[hydroxy(phenylmethoxy)phosphinyl]-L-leucyl-N-[(1S)-1-formyl-3-methylbutyl]-),

PhCH2O—P(OH)(O)-Phe-Ala-Leu-H (L-Alaninamide, N-[hydroxy(phenylmethoxy)phosphinyl]-L-phenylalanyl-N-[(1S)-1-formyl-3-methylbutyl]-), or

MeO—P(OH)(O)-Leu-Gly-Ala-Leu-H; (L-Alaninamide, N-(hydroxymethoxyphosphinyl)-L-leucylglycyl-N-[(1S)-1-formyl-3-methylbutyl]-).

A preferred example is Cbz-Gly-Ala-Tyr-H.

Further examples of such peptide aldehydes include

α-MAPI (3,5,8,11-Tetraazatridecanoic acid, 6-[3-[(aminoiminomethyl)amino]propyl]-12-formyl-9-(1-methylethyl)-4,7,10-trioxo-13-phenyl-2-(phenylmethyl)-, (2S,6S,9S,12S)-

L-Valinamide, N2-[[(1-carboxy-2-phenylethyl)amino]carbonyl]-L-arginyl-N-(1-formyl-2-phenylethyl)-, [1(S),2(S)]—; L-Valinamide, N2-[[[(1S)-1-carboxy-2-phenylethyl]amino]carbonyl]-L-arginyl-N-[(1S)-1-formyl-2-phenylethyl]- (9Cl); SP-Chymostatin B),

β-MAPI (L-Valinamide, N2-[[[(1S)-1-carboxy-2-phenylethyl]amino]carbonyl]-L-arginyl-N-[(1R)-1-formyl-2-phenylethyl]-L-Valinamide, N2-[[(1-carboxy-2-phenylethyl)amino]carbonyl]-L-arginyl-N-(1-formyl-2-phenylethyl)-, [1(S),2(R)]—),

Phe-C(═O)-Arg-Val-Tyr-H (L-Valinamide, N2-[[[(1S)-1-carboxy-2-phenylethyl]amino]carbonyl]-L-arginyl-N-[(1S)-1-formyl-2-(4-hydroxyphenyl)ethyl]- (9Cl)),

Phe-C(═O)-Gly-Gly-Tyr-H, (3,5,8,11-Tetraazatridecanoic acid, 12-formyl-13-(4-hydroxyphenyl)-4,7,10-trioxo-2-(phenylmethyl)-, (2S,12S)—),

Phe-C(═O)-Gly-Ala-Phe-H, (3,5,8,11-Tetraazatridecanoic acid, 12-formyl-9-methyl-4,7,10-trioxo-13-phenyl-2-(phenylmethyl)-, (2S,9S,12S)—),

Phe-C(═O)-Gly-Ala-Tyr-H (3,5,8,11-Tetraazatridecanoic acid, 12-formyl-13-(4-hydroxyphenyl)-9-methyl-4,7,10-trioxo-2-(phenylmethyl)-, (2S,9S,12S)—),

Phe-C(═O)-Gly-Ala-Leu-H, (3,5,8,11-Tetraazapentadecanoic acid, 12-formyl-9,14-dimethyl-4,7,10-trioxo-2-(phenylmethyl)-, (2S,9S,12S)—),

Phe-C(═O)-Gly-Ala-Nva-H, (3,5,8,11-Tetraazapentadecanoic acid, 12-formyl-9-methyl-4,7,10-trioxo-2-(phenylmethyl)-, (2S,9S,12S)—),

Phe-C(═O)-Gly-Ala-Nle-H (3,5,8,11-Tetraazahexadecanoic acid, 12-formyl-9-methyl-4,7,10-trioxo-2-(phenylmethyl)-, (2S,9S,12S)—),

Tyr-C(═O)-Arg-Val-Tyr-H (L-Valinamide, N2-[[[(1S)-1-carboxy-2-(4-hydroxyphenyl)ethyl]amino]carbonyl]-L-arginyl-N-[(1S)-1-formyl-2-(4-hydroxyphenyl)ethyl]- (9Cl))

Tyr-C(═O)-Gly-Ala-Tyr-H (3,5,8,11-Tetraazatridecanoic acid, 12-formyl-13-(4-hydroxyphenyl)-2-[(4-hydroxyphenyl)methyl]-9-methyl-4,7,10-trioxo-, (2S,9S,12S)—)

Phe-C(═S)-Arg-Val-Phe-H, (3,5,8,11-Tetraazatridecanoic acid, 6-[3-[(aminoiminomethyl)amino]propyl]-12-formyl-9-(1-methylethyl)-7,10-dioxo-13-phenyl-2-(phenylmethyl)-4-thioxo-, (2S,6S,9S,12S)—),

Phe-C(═S)-Arg-Val-Tyr-H, (3,5,8,11-Tetraazatridecanoic acid, 6-[3-[(aminoiminomethyl)amino]propyl]-12-formyl-13-(4-hydroxyphenyl)-9-(1-methylethyl)-7,10-dioxo-2-(phenylmethyl)-4-thioxo-, (2S,6S,9S,12S)—),

Phe-C(═S)-Gly-Ala-Tyr-H, (3,5,8,11-Tetraazatridecanoic acid, 12-formyl-13-(4-hydroxyphenyl)-9-methyl-7,10-dioxo-2-(phenylmethyl)-4-thioxo-, (2S,9S,12S)—),

Antipain (L-Valinamide, N2-[[(1-carboxy-2-phenylethyl)amino]carbonyl]-L-arginyl-N-[4-[(aminoiminomethyl)amino]-1-formylbutyl]-),

GE20372A (L-Valinamide, N2-[[[(1S)-1-carboxy-2-(4-hydroxyphenyl)ethyl]amino]carbonyl]-L-arginyl-N-[(1S)-1-formyl-2-phenylethyl]-

L-Valinamide, N2-[[[1-carboxy-2-(4-hydroxyphenyl)ethyl]amino]carbonyl]-L-arginyl-N-(1-formyl-2-phenylethyl)-, [1 (S),2(S)]—),

GE20372B (L-Valinamide, N2-[[[(1S)-1-carboxy-2-(4-hydroxyphenyl)ethyl]amino]carbonyl]-L-arginyl-N-[(1R)-1-formyl-2-phenylethyl]-

L-Valinamide, N2-[[[1-carboxy-2-(4-hydroxyphenyl)ethyl]amino]carbonyl]-L-arginyl-N-(1-formyl-2-phenylethyl)-, [1(S),2(R)]—),

Chymostatin A (L-Leucinamide, (2S)-2-[(4S)-2-amino-3,4,5,6-tetrahydro-4-pyrimidinyl]-N-[[[(1S)-1-carboxy-2-phenylethyl]amino]carbonyl]glycyl-N-(1-formyl-2-phenylethyl)-

L-Leucinamide, (2S)-2-[(4S)-2-amino-1,4,5,6-tetrahydro-4-pyrimidinyl]-N-[[[(1S)-1-carboxy-2-phenylethyl]amino]carbonyl]glycyl-N-(1-formyl-2-phenylethyl)- (9Cl); L-Leucinamide, L-2-(2-amino-1,4,5,6-tetrahydro-4-pyrimidinyl)-N-[[(1-carboxy-2-phenylethyl)amino]carbonyl]glycyl-N-(1-formyl-2-phenylethyl)-, stereoisomer),

Chymostatin B (L-Valinamide, (2S)-2-[(4S)-2-amino-3,4,5,6-tetrahydro-4-pyrimidinyl]-N-[[[(1S)-1-carboxy-2-phenylethyl]amino]carbonyl]glycyl-N-(1-formyl-2-phenylethyl)-

L-Valinamide, (2S)-2-[(4S)-2-amino-1,4,5,6-tetrahydro-4-pyrimidinyl]-N-[[[(1S)-1-carboxy-2-phenylethyl]amino]carbonyl]glycyl-N-(1-formyl-2-phenylethyl)- (9Cl); L-Valinamide, L-2-(2-amino-1,4,5,6-tetrahydro-4-pyrimidinyl)-N-[[(1-carboxy-2-phenylethyl)amino]carbonyl]glycyl-N-(1-formyl-2-phenylethyl)-, stereoisomer), and

Chymostatin C (L-Isoleucinamide, (2S)-2-[(4S)-2-amino-3,4,5,6-tetrahydro-4-pyrimidinyl]-N-[[[(1S)-1-carboxy-2-phenylethyl]amino]carbonyl]glycyl-N-(1-formyl-2-phenylethyl)-

L-Isoleucinamide, (2S)-2-[(4S)-2-amino-1,4,5,6-tetrahydro-4-pyrimidinyl]-N-[[[(1S)-1-carboxy-2-phenylethyl]amino]carbonyl]glycyl-N-(1-formyl-2-phenylethyl)- (9Cl); L-Isoleucinamide, L-2-(2-amino-1,4,5,6-tetrahydro-4-pyrimidinyl)-N-[[(1-carboxy-2-phenylethyl)amino]carbonyl]glycyl-N-(1-formyl-2-phenylethyl)-, stereoisomer).

Peptide Aldehyde Adducts

Instead of a peptide aldehyde, the protease inhibitor may be an adduct of a peptide aldehyde. The adduct maybe a hydrosulfite adduct having the formula P-(A)_{y}-L-(B)ₓ—N(H)—CHR—CH(OH)—SO₃M, wherein P, A, y, L, B, x and R are defined as above, and M is H or an alkali metal, preferably Na or K. Alternatively, the adduct may be a hemiacetal having the formula P-(A)_{y}-L-(B)ₓ—N(H)—CHR—CH(OH)—OR, wherein P, A, y, L, B, x and R are defined as above. A preferred embodiment is a hydrosulfite adduct wherein P=Cbz, B²=Gly; B¹=Ala; B⁰=Tyr (so R=PhCH₂, R′═OH), x=2, y=0, L=A=absent and M=Na (Cbz-Gly-Ala-N(H)—CH(CH₂-p-C₆H₄OH)—CH(OH)—SO₃Na, L-Alaninamide, N-[(phenylmethoxy)carbonyl]glycyl-N-[2-hydroxy-1-[(4-hydroxyphenyl)methyl]-2-sulfoethyl]-, sodium salt (1:1)).

The general formula of the hydrosulfite adduct of a peptide aldehyde may also be written: P-A²-A¹-L-B³—B²—B¹—N(H)—CHR—CH(OH)—SO₃M, where P, A², A¹,L, B³, B², B¹, R and M are as defined above.

Alternatively, the adduct of a peptide aldehyde can be Cbz-Gly-Ala-N(H)—CH(CH₂-p-C₆H₄OH)—CH(OH)—SO₃Na (Sodium (2S)—[(N—{N-[(benzyloxy)carbonyl]glycyl}-L-alaninyl)amino]-1-hydroxy-3-(4-hydroxyphenyl)propane-1-sulfonate) or Cbz-Gly-Ala-N(H)—CH(CH2Ph)-CH(OH)—SO₃Na (Sodium (2S)—[(N—{N-[(benzyloxy)carbonyl]glycyl}-L-alaninyl)amino]-1-hydroxy-3-(phenyl)propane-1-sulfonate) or “MeO-CO_Val-Ala-N(H)—CH(CH2CH(CH₃)₂)—CH(OH)—SO₃Na (Sodium (2S)—[(N—{N-[(benzyloxy)carbonyl]glycyl}-L-alaninyl)amino]-1-hydroxy-3-(2-propanyl)propane-1-sulfonate).

Other preferred peptide aldehyde bisulfites are

Cbz-Arg-Ala-NHCH(CH₂C6H₄OH)C(OH)(SO₃M)-H where M=Na,

Ac-Gly-Ala-NHCH(CH₂C6H₄OH)C(OH)(SO₃M)-H, where M=Na,

Cbz-Gly-Ala-NHCH(CH₂C6H₄OH)C(OH)(SO₃M)-H, where M=Na (L-Alaninamide, N-[(phenylmethoxy)carbonyl]glycyl-N-[2-hydroxy-1-[(4-hydroxyphenyl)methyl]-2-sulfoethyl]-, sodium salt (1:1)),

Cbz-Gly-Ala-NHCH(CH₂CH(CH₃)₂))C(OH)(SO₃M)-H, where M=Na,

Cbz-Val-Ala-NHCH(CH₂CH(CH₃)₂))C(OH)(SO₃M)-H, where M=Na,

Cbz-Gly-Ala-NHCH(CH₂Ph)C(OH)(SO₃M)-H, where M=Na,

Cbz-Gly-Ala-NHCH(CH(CH₃)₂)C(OH)(SO₃M)-H, where M=Na,

Cbz-Gly-Gly-NHCH(CH₂C₆H₄OH)C(OH)(SO₃M)-H, where M=Na,

Cbz-Gly-Gly-NHCH(CH₂Ph)C(OH)(SO₃M)-H, where M=Na,

Cbz-Arg-Val-NHCH(CH₂C₆H₄OH)C(OH) (SO₃M)-H, where M=Na,

Cbz-Leu-Val-NHCH(CH₂C6H₄OH)C(OH)(SO₃M)-H, where M=Na,

Ac-Leu-Gly-Ala-NHCH(CH₂C₆H₄OH)C(OH)(SO₃M)-H, where M=Na,

Ac-Phe-Gly-Ala-NHCH(CH₂C₆H₄OH)C(OH)(SO₃M)-H, where M=Na,

Ac-Tyr-Gly-Ala-NHCH(CH₂C₆H₄OH)C(OH)(SO₃M)-H, where M=Na,

Ac-Phe-Gly-Ala-NHCH(CH₂CH(CH₃)₂))C(OH)(SO₃M)-H, where M=Na,

Ac-Phe-Gly-Ala-NHCH(CH₂Ph)C(OH)(SO₃M)-H, where M=Na,

Ac-Phe-Gly-Val-NHCH(CH₂C₆H₄OH)C(OH)(SO₃M)-H, where M=Na,

Ac-Phe-Gly-Ala-NHCH(CH₂CH₂SCH₃)(SO₃M)-H, where M=Na,

Ac-Trp-Leu-Val-NHCH(CH₂C₆H₄OH)C(OH)(SO₃M)-H, where M=Na,

MeO—CO-Val-Ala-NHCH(CH₂CH(CH₃)₂))C(OH)(SO₃M)-H, where M=Na,

MeNCO-Val-Ala-NHCH(CH₂CH(CH₃)₂))C(OH)(SO₃M)-H, where M=Na,

MeO—CO-Phe-Gly-Ala-NHCH(CH₂CH(CH₃)₂))C(OH)(SO₃M)-H, where M=Na,

MeO—CO-Phe-Gly-Ala-NHCH(CH₂Ph)C(OH)(SO₃M)-H, where M=Na,

MeSO₂-Phe-Gly-Ala-NHCH(CH₂CH(CH₃)₂))C(OH)(SO₃M)-H, where M=Na,

MeSO₂—Val-Ala-NHCH(CH₂CH(CH₃)₂))C(OH)(SO₃M)-H, where M=Na,

PhCH₂O(OH)(O)P-Val-Ala-NHCH(CH₂CH(CH₃)₂))C(OH)(SO₃M)-H, where M=Na,

EtSO₂-Phe-Gly-Ala-NHCH(CH₂CH(CH₃)₂))C(OH)(SO₃M)-H, where M=Na,

PhCH₂SO₂—Val-Ala-NHCH(CH₂CH(CH₃)₂))C(OH)(SO₃M)-H, where M=Na,

PhCH₂O(OH)(O)P-Leu-Ala-NHCH(CH₂CH(CH₃)₂))C(OH)(SO₃M)-H, where M=Na,

PhCH₂O(OH)(O)P-Phe-Ala-NHCH(CH₂CH(CH₃)₂))C(OH)(SO₃M)-H, where M=Na,

MeO(OH)(O)P-Leu-Gly-Aa-NHCH(CH₂CH(CH₃)₂))C(OH)(SO₃M)-H, where M=Na,and

Phe-urea-Arg-Val-NHCH(CH₂C6H₄OH)C(OH)(SO₃M)-H where M=Na.

### Salt

The salt used in the bar is a salt of a monovalent cation and an organic anion. The monovalent cation may be for example Na⁺, K⁺ or NH₄⁺. The organic anion may be for example formate, acetate, citrate or lactate. Thus a salt of a monovalent cation and an organic anion may be, for example, sodium formate, potassium formate, ammonium formate, sodium acetate, potassium acetate, ammonium acetate, sodium lactate, potassium lactate, ammonium lactate, mono-sodium citrate, di-sodium citrate, tri-sodium citrate, sodium potassium citrate, potassium citrate, ammonium citrate or the like. A particular embodiment is sodium formate.

### Enzymes of the Present Invention

In one embodiment of the present invention, the polypeptide of the present invention may be added to a warewashing detergent composition in an amount corresponding to 0.001-200 mg of protein, such as 0.005-100 mg of protein, preferably 0.01-50 mg of protein, more preferably 0.05-20 mg of protein, even more preferably 0.1-10 mg of protein per liter of wash liquor.

The enzyme(s) of the warewashing detergent composition of the invention may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formylphenyl boronic acid, and the composition may be formulated as described above. In a preferred embodiment the enzyme(s) is stabilized using a protease inhibitor.

### Surfactants

The detergent composition comprises one or more surfactants, of which at least one surfactant is anionic. Other surfactants may be anionic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.0% to 10% by weight, such as about 0.1% to about 5%, or about 0% to about 3%, or about 0% to about 2%. The surfactant(s) is chosen based on the desired cleaning application, and may include any conventional surfactant(s) known in the art.

When included therein, the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein, the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

When included therein, the detergent will usually contain from about 0% to about 40% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein, the detergent will usually contain from about 0% to about 40% by weight of a zwitterionic surfactant. Non-limiting examples of zwitterionic surfactants include betaines such as alkyldimethylbetaines, sulfobetaines, and combinations thereof.

### Builders and Co-Builders

The detergent composition may contain about 0-65% by weight, such as about 5% to about 50% of a detergent builder or co-builder, or a mixture thereof. The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis(methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), a-alanine-N,N-diacetic acid (a-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053

### Zeolites

A preferred class of zeolites is characterized as “intermediate” silicate/aluminate zeolites. The intermediate zeolites are characterized by SiOx/A10z molar ratios of less than about 10. Preferably the molar ratio of Si02/A102 ranges from about 2 to about 10. The intermediate zeolites can have an advantage over the “high” zeolites. The intermediate zeolites have a higher affinity for amine-type odors, they are more weight efficient for odor absorption because they have a larger surface area, and they are more moisture tolerant and retain more of their odor absorbing capacity in water than the high zeolites. A wide variety of intermediate zeolites suitable for use herein are commercially available as Valfor@ CP301-68, Valfor@ 300-63, Valfor@ CP300-35, and Valfor® CP300-56, available from PQ Corporation, and the CBV100® series of zeolites from Conteka.

Zeolite materials marketed under the trade name Absents® and Smellrite®, available from The Union Carbide Corporation and UOP are also preferred. Such materials are preferred over the intermediate zeolites for control of sulfur-containing odors, e.g. thiols, mercaptans.

When zeolites are used as odor control agents in compositions that are to be sprayed onto surfaces, the zeolite material preferably has a particle size of less than about 10 microns and is present in the composition at a level of less than about 1% by weight of the composition.

### Bleaching Systems

The detergent may contain 0-30% by weight, such as about 1% to about 20%, of a bleaching system. Any bleaching system known in the art for use in detergents may be utilized. Suitable bleaching system components include bleaching catalysts, photobleaches, bleach activators, sources of hydrogen peroxide such as sodium percarbonate, sodium perborates and hydrogen peroxide-urea (1:1), preformed peracids and mixtures thereof. Suitable preformed peracids include, but are not limited to, peroxycarboxylic acids and salts, diperoxydicarboxylic acids, perimidic acids and salts, peroxymonosulfuric acids and salts, for example, Oxone (R), and mixtures thereof. Non-limiting examples of bleaching systems include peroxide-based bleaching systems, which may comprise, for example, an inorganic salt, including alkali metal salts such as sodium salts of perborate (usually mono- or tetra-hydrate), percarbonate, persulfate, perphosphate, persilicate salts, in combination with a peracid-forming bleach activator. The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable examples are tetraacetylethylenediamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A particular family of bleach activators of interest was disclosed in EP624154 and particulary preferred in that family is acetyl triethyl citrate (ATC). ATC or a short chain triglyceride like triacetin has the advantage that it is environmentally friendly Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). The bleaching system may also include a bleach catalyst. In some embodiments the bleach component may be an organic catalyst selected from the group consisting of organic catalysts having the following formulae:

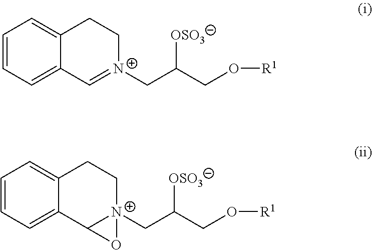

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl. Other exemplary bleaching systems are described, e.g. in WO2007/087258, WO2007/087244, WO2007/087259, EP1867708 (Vitamin K) and WO2007/087242. Suitable photobleaches may for example be sulfonated zinc or aluminium phthalocyanines.

Preferably, the bleach component comprises a source of peracid in addition to bleach catalyst, particularly organic bleach catalyst. The source of peracid may be selected from (a) preformed peracid; (b) percarbonate, perborate or persulfate salt (hydrogen peroxide source) preferably in combination with a bleach activator; and (c) perhydrolase enzyme and an ester for forming peracid in situ in the presence of water in a treatment step.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated.

### Hueing Agents

The detergent compositions of the present invention may also include hueing agents such as dyes or pigments, which when formulated in detergent compositions can deposit onto a surface when said surface is contacted with a wash liquor comprising said detergent compositions and thus altering the tint of said surface through absorption/reflection of visible light. Fluorescent whitening agents emit at least some visible light. In contrast, hueing agents alter the tint of a surface as they absorb at least a portion of the visible light spectrum. Suitable hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO2005/03274, WO2005/03275, WO2005/03276 and EP1876226 (hereby incorporated by reference). The detergent composition preferably comprises from about 0.00003 wt % to about 0.2 wt %, from about 0.00008 wt % to about 0.05 wt %, or even from about 0.0001 wt % to about 0.04 wt % hueing agent. The composition may comprise from 0.0001 wt % to 0.2 wt % hueing agent, this may be especially preferred when the composition is in the form of a unit dose pouch. Suitable hueing agents are also disclosed in, e.g. WO 2007/087257 and WO2007/087243.

### Enzymes

The warewashing detergent composition and/or the wash liquor may comprise one or more additional enzymes such as an additional protease, lipase, cutinase, an additional amylase, carbohydrase, cellulase, pectinase, mannanase, arabinase, galactanase, xylanase, oxidase, e.g., a laccase, and/or peroxidase.

In general the properties of the selected enzyme(s) should be compatible with the selected detergent, (i.e., pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO99/001544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, and Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 1999/064619. A commercially available mannanase is Mannaway (Novozymes A/S).

Cellulase

Suitable cellulases include complete cellulases or mono-component endoglucanases of bacterial or fungal origin. Chemically or genetically modified mutants are included. The cellulase may for example be a mono-component or a mixture of mono-component endo-1,4-beta-glucanase often just termed endoglucanases. Suitable cellulases include a fungal cellulase from Humicola insolens (U.S. Pat. No. 4,435,307) or from Trichoderma, e.g. T. reesei or T. viride. Examples of cellulases are described in EP 0 495 257. Other suitable cellulases are from Thielavia e.g. Thielavia terrestris as described in WO 96/29397 or Fusarium oxysporum as described in WO 91/17244 or from Bacillus as described in, WO 02/099091 and JP 2000210081. Other examples are cellulase variants such as those described in WO 94/07998, EP 0 531 315, U.S. Pat. Nos. 5,457,046, 5,686,593, 5,763,254, WO 95/24471, WO 98/12307 Commercially available cellulases include Carezyme®, Celluzyme®, Celluclean®, Celluclast® and Endolase®; Renozyme®; Whitezyme® (Novozymes A/S) Puradax®, Puradax HA, and Puradax EG (available from Genencor).

Proteases

Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus Lentus protease shown in SEQ ID NO 1 of WO 2016/001449. More preferred the subtilase variants may comprise the mutations: S3T, V41, S9R, S9E, A15T, S24G, S24R, K27R, N42R, S55P, G59E, G59D, N60D, N60E, V66A, N74D, N85S, N85R, G96S, G96A, S97G, S97D, S97A, S97SD, S99E, S99D, S99G, S99M, S99N, S99R, S99H, S101A, V1021, V102Y, V102N, S104A, G116V, G116R, H118D, H118N, N120S, S126L, P127Q, S128A, S154D, A156E, G157D, G157P, S158E, Y161A, R164S, Q176E, N179E, S182E, Q185N, A188P, G189E, V193M, N198D, V1991, Y203W, S206G, L211Q, L211D, N212D, N212S, M216S, A226V, K229L, Q230H, Q239R, N246K, N255W, N255D, N255E, L256E, L256D T268A, R269H. The protease variants are preferably variants of the Bacillus Lentus protease (Savinase®) shown in SEQ ID NO 1 of WO 2016/001449, the Bacillus amylolichenifaciens protease (BPN′) shown in SEQ ID NO 2 of WO2016/001449. The protease variants preferably have at least 80% sequence identity to SEQ ID NO 1 or SEQ ID NO 2 of WO 2016/001449.

A protease variant comprising a substitution at one or more positions corresponding to positions 171, 173, 175, 179, or 180 of SEQ ID NO: 1 of WO2004/067737, wherein said protease variant has a sequence identity of at least 75% but less than 100% to SEQ ID NO: 1 of WO2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity@ 100T, Blaze Evity@ 125T, Blaze Evity@ 150T, Neutrase®, Everlase® and Esperase® (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser@, Preferenz P100, Purafect Prime®, Preferenz P110™, Effectenz P1000, Purafect®™, Effectenz P1050, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

Lipases and Cutinases:

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412), Geobacillus stearothermophilus lipase (WO11/084417), lipase from Bacillus subtilis (WO11/084599), and lipase from Streptomyces griseus (WO11/150157) and S. pristinaespiralis (WO12/137147). Other examples are lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g. acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

Amylases:

Suitable amylases which can be used in the present invention may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:

M197T;

H156Y+A181T+N190F+A209V+Q264S; or

G48A+T491+G107A+H156Y+A181T+N190F+1201F+A209V+Q264S.

Further amylases which are suitable are amylases having SEQ ID NO: 6 in WO 99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 08/153815, SEQ ID NO: 10 in WO 01/66712 or variants thereof having 90% sequence identity to SEQ ID NO: 2 of WO 08/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:

N128C+K178L+T182G+Y305R+G475K;

N128C+K178L+T182G+F202Y+Y305R+D319T+G475K;

S125A+N128C+K178L+T182G+Y305R+G475K; or

S125A+N128C+T131I+T165I+K178L+T182G+Y305R+G475K wherein the variants are C-terminally truncated and optionally further comprises a substitution at position 243 and/or a deletion at position 180 and/or position 181.

Further suitable amylases are amylases having SEQ ID NO: 1 of WO13184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181. Most preferred amylase variants of SEQ ID NO: 1 are those having the substitutions:

E187P+1203Y+G476K

E187P+1203Y+R458N+T459S+D460T+G476K

wherein the variants optionally further comprises a substitution at position 241 and/or a deletion at position 178 and/or position 179.

Further suitable amylases are amylases having SEQ ID NO: 1 of WO10104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:

N21 D+D97N+V128I

wherein the variants optionally further comprises a substitution at position 200 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Fungamyl™, Stainzyme™, Stainzyme Plus™, Natalase™, Liquozyme X and BAN′ (from Novozymes A/S), and Rapidase™ Purastar™/Effectenz™, Powerase, Preferenz S1000, Preferenz S100 and Preferenz S110 (from Genencor International Inc./DuPont).

Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257. Commercially available peroxidases include Guardzyme™ (Novozymes A/S).

A peroxidase according to the invention also include a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions.

In an embodiment, the haloperoxidase of the invention is a chloroperoxidase. Preferably, the haloperoxidase is a vanadium haloperoxidase, i.e., a vanadate-containing haloperoxidase. In a preferred method of the present invention the vanadate-containing haloperoxidase is combined with a source of chloride ion.

Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

In an preferred embodiment, the haloperoxidase is derivable from Curvularia sp., in particular Curvularia verruculosa or Curvularia inaequalis, such as C. inaequalis CBS 102.42 as described in WO 95/27046; or C. verruculosa CBS 147.63 or C. verruculosa CBS 444.70 as described in WO 97/04102; or from Drechslera hartlebii as described in WO 01/79459, Dendryphiella salina as described in WO 01/79458, Phaeotrichoconis crotalarie as described in WO 01/79461, or Geniculosporium sp. as described in WO 01/79460.

An oxidase according to the invention include, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5).

Preferred laccase enzymes are enzymes of microbial origin. The enzymes may be derived from plants, bacteria or fungi (including filamentous fungi and yeasts).

Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885).

Suitable examples from bacteria include a laccase derivable from a strain of Bacillus.

A laccase derived from Coprinopsis or Myceliophthora is preferred; in particular a laccase derived from Coprinopsis cinerea, as disclosed in WO 97/08325; or from Myceliophthora thermophila, as disclosed in WO 95/33836.

### Formulation of Enzyme in Co-Granule

The enzyme(s) of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co- granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component. WO 2013/188331 also relates to a method of treating and/or cleaning a surface, preferably a fabric surface comprising the steps of (i) contacting said surface with the detergent composition as claimed and described herein in an aqueous wash liquor, (ii) rinsing and/or drying the surface.

The multi-enzyme co-granule may comprise an enzyme of the invention and (a) one or more enzymes selected from the group consisting of first- wash lipases, cleaning cellulases, xyloglucanases, perhydrolases, peroxidases, lipoxygenases, laccases and mixtures thereof; and (b) one or more enzymes selected from the group consisting of hemicellulases, proteases, care cellulases, cellobiose dehydrogenases, xylanases, phospholipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

The invention is further defined in the following paragraphs:
1. Use of an amylase and/or a protease for removing and/or reducing soil on a surface, and/or for reducing redeposition, wherein the surface is exposed to the amylase and/or protease for a time period of 10 to 240 seconds.
2. Use according to paragraph 1, wherein the surface is exposed to the amylase and/or protease for a time period of 10 to 220 seconds, such in the range of 10 to 200 seconds, in the range of 10 to 180 seconds, in the range of 10 to 160 seconds, in the range of 10 to 140 seconds, in the range of 10 to 120 seconds, in the range of 10 to 100 seconds, in the range of 10 to 80 seconds, in the range of 10 to 70 seconds, in the range of 10 to 66 seconds, in the range of 20 to 66 seconds, in the range of 25 to 66 seconds, in the range of 28 to 66 seconds, in the range of 28 to 60 seconds, in the range of 28 to 55 seconds, in the range of 28 to 50 seconds or in the range of 28 to 45 seconds.
3. Use according to any of the preceding paragraphs, wherein the amylase and/or protease is comprised in a liquid having a pH in the range of 7-10.5.
4. Use according to paragraph 4, wherein the pH is in the range of 7.5-10.5, such as in the range of 7.5-10, in the range of 7.5-9.5, in the range of 7.5-9.0, in the range of 7.5-8.5, in the range of 7.5-8.2, or in the range of 7.8-8.2.
5. Use according to any of the preceding paragraphs, wherein an amylase is used.
6. Use according to paragraph 5, wherein the amylase has a sequence identity of at least 70%, such as at least 75%, such as at least 80%, such as at least 85%, such as at least 90%, such as at least 95%, such as at least 98%, such as at least 99%, such as 100%, to the sequence of SEQ ID NO: 1, 2, 3 or 4.
7. Use according to paragraph 6, wherein the amylase is an alpha-amylase variant comprises the modifications D183*+G184*+R118K+N195F+R320K+R458K or M9L+R118K+G149A+G182T+G186A+D183*+G184*+N195F+M202L+T2571+Y295F+N299Y+R320K+M323T+A339S+E345R+R458K of SEQ ID NO: 1; D183*+G184* or W140Y+D183*+G184*+N195F+V206Y+Y243F+E260G+G304R+G476K of SEQ ID NO: 2; H156Y+A181T+N190F+A209V+Q264S of SEQ ID NO: 3, wherein said alpha-amylase variant has a sequence identity of at least 75% but less than 100% to SEQ ID NO: 1, 2 or 3, respectively, and wherein said alpha-amylase variant has alpha-amylase activity.
8. Use according to paragraph 6, wherein the amylase is an alpha-amylase variant comprising a modification in one or more positions corresponding to positions H1, N54, V56, K72, G109, F113, R116, T134, W140, W159, W167, Q169, Q172, L173, A174, R181, G182, D183, G184, W189, E194, N195, V206, G255, N260, F262, A265, W284, F289, S304, G305, W347, K391, Q395, W439, W469, R444, F473, G476, and G477 of SEQ ID NO: 4, wherein said alpha-amylase variant has a sequence identity of at least 75% but less than 100% to SEQ ID NO: 4 and wherein said alpha-amylase variant has alpha-amylase activity.
9. Use according to paragraph 6 or 8, wherein said modification in one or more positions is selected from the group consisting of: H1*, H1A, N54S, V56T, K72R, G109A, F113Q, R116Q, R116H, T134E, W140Y, W140F, W140H, W159Y, W159F, W159H, W167Y, W167H, W167F, Q169E, Q172K, Q172G, Q172N, L173P, A174*, A174S, R181*, G182*, D183*, G184*, G184T, W189Y, W189F, W189H, W189E, W189D, W189Q, W189N, E194D, E194N, E194S, N195F, V206L, V206F, V206Y, G255A, N260G, N260P, N260A, N260G, N260P, N260A, A265G, W284G, W284H, F289H, S304K, S304R, S304Q, S304E, G305K, G305R, G305Q, G305E, W347Y, W347F, W347H, K391A, Q395P, W439N, W439Q, W439T, R444Q, W469T, W469N, F473R, G476R, G476Q, G476E, G476K G477K, G477R, G477Q, and G477E wherein the positions correspond to positions of SEQ ID NO: 4.
10. Use according to paragraph 6 or 8-9, wherein said at least one alpha-amylase variant comprises a deletion in the positions corresponding to R181+G182; R181+D183; R181+G184; G182+D183; G182+G184; or D183+G184 of SEQ ID NO:4.
11. Use according to paragraph 6 or 8-10, wherein said alpha-amylase variant is selected from the group consisting of:
H1*+N54S+V56T+G109A+Q169E+Q172K+A174*+G182*+D183*+N195F+V206L+K391A+G476K;
H1*+N54S+V56T+G109A+R116H+A174S+G182*+D183*+N195F+V206L+K391A+G476K;
H1*+N54S+V56T+K72R+G109A+F113Q+R116Q+W167F+Q172G+A174S+G182*+D183*+G184T+N195F+V206L+K391A+P473R+G476K;
H1*+N54S+V56T+G109A+F113Q+R116Q+Q172N+A174S+G182*+D183*+N195F+V206L+A265G+K391A+P473R+G476K;
H1*+N54S+V56T+K72R+G109A+F113Q+W167F+Q172R+A174S+G182*+D183*+N195F+V206L+K391A+G476K;
H1*+N54S+V56T+K72R+G109A+R116H+T134E+W167F+Q172G+L173V+A174S+G182*+D183*+N195F+V206L+G255A+K391A+G476K;
H1*+N54S+V56T+K72R+G109A+R116H+T134E+W167F+Q172G+L173V+A174S+G182*+D183*+N195F+V206L+G255A+K391A+Q395P+T444Q+P473R+G476K;
H1*+N54S+V56T+G109A+T134E+A174S+G182*+D183*+N195F+V206L+K391A+G476K;
H1*+N54S+V56T+K72R+G109A+A174S+G182*+D183*+N195F+V206L+G255A+K391A+G476K;
H1*+N54S+V56T+K72R+G109A+F113Q+R116Q+W167F+Q172G+A174S+G184T+N195F+V206L+K391A+P473R+G476K, and H1*+N54S+V56T+G109A+W167F+Q172E+L173P+A174K+G182*+D183*+N195F+V206L+K391A+G476K, of the polypeptide of SEQ ID NO: 4 and wherein said alpha-amylase variant shares at least 80%, such as at least 85%, such as at least 90%, such as at least 93%, such as at least 94%, such as at least 95%, such as at least 96%, such as at least 97%, such as at least 98%, but less than 100% sequence identity with the polypeptide of SEQ ID NO: 4 and wherein said alpha-amylase variant has alpha-amylase activity.
12. Use according to any of the preceding paragraphs, wherein a protease is used.
13. Use according to paragraph 12, wherein the protease is selected from the group consisting of
   i. a protease having a sequence identity of at least 70%, such as at least 75%, such as at least 80%, such as at least 85%, such as at least 90%, such as at least 95%, such as at least 98%, such as at least 99%, such as 100%, to the sequences of SEQ ID NOs: 5, 6, 7 or 8;
   ii. a protease variant comprising a substitution at one or more positions corresponding to positions 9, 15, 36, 61, 68, 76, 99, 106, 120, 167, 170, 194, 195, 205, 218, 235, 245 or 261 of SEQ ID NO: 3, wherein said protease variant has a sequence identity of at least 75% but less than 100% to SEQ ID NO: 5,
   iii. The protease of SEQ ID NO: 24.
14. Use according to paragraph 13, wherein the protease is selected from the group consisting of: M222S, *36D+N76D+N120D+G195E+K235L, Y167A+R170S+A194P, S99SE, V68A+S106A, S9R+A15T+V68A+N218D+Q245R, S9R+A15T+G61E+V68A+A194P+V205I+Q245R+N261D and S99AD of SEQ ID NO: 5, wherein said protease variant has a sequence identity of at least 75% but less than 100% to SEQ ID NO: 5 and wherein said protease variant has protease activity.
15. Use according to any of the preceding paragraphs, wherein an amylase and a protease is used.
16. Use according to any of the preceding paragraphs, wherein an additional enzyme is used, which enzyme is selected from the group consisting of a protease, lipase, cutinase, an amylase, carbohydrase, cellulase, pectinase, mannanase, arabinase, galactanase, xylanase, oxidase, a licheninase, a laccase and/or peroxidase.
17. Use according to any of the preceding paragraphs, wherein the surface is the inner surface of a warewashing machine or the surface of a ware.
18. Warewashing detergent composition comprising an amylase and/or a protease and one or more detergent components which composition has a pH in the range of 7-10.5.
19. Composition according to paragraph 18, wherein the detergent component is selected from the group consisting of as surfactants, builders, chelators or chelating agents, bleach system or bleach components, polymers, foam boosters, suds suppressors, dyes, perfume, tannish inhibitors, optical brighteners, bactericides, fungicides, soil suspending agents, anti-corrosion agents, enzyme stabilizers, enzyme inhibitors or activators, transferase(s), hydrolytic enzymes, oxido reductases, bluing agents and fluorescent dyes, antioxidant, and solubilizers.
20. Composition according to any of the paragraphs 18-19, wherein the composition comprises at least 5% wt. of a chelator.
21. Composition according to any of the paragraphs 18-20, wherein the composition comprises less than 2% of a surfactant.
22. Composition according to any of the paragraphs 18-20, wherein the composition does not comprise a surfactant.
23. Composition according to any of the paragraphs 18-22, wherein the composition is a warewashing detergent composition for industrial or institutional use.
24. Composition according to any of the paragraphs 18-23, wherein the pH of the wash liquor is in the range of 7-10.5.
25. Composition according to paragraph 24, wherein the pH is in the range of 7.5-10.5, such as in the range of 7.5-10, in the range of 7.5-9.5, in the range of 7.5-9.0, in the range of 7.5-8.5, in the range of 7.5-8.2, or in the range of 7.8-8.2.
26. Composition according to any of the paragraphs 18-25, wherein the composition is a bar, a block a homogenous tablet, a tablet having two or more layers, a pouch having one or more compartments, a regular or compact powder, a granule, a paste, a gel, or a regular, compact or concentrated liquid.
27. Composition according to any of the paragraphs 18-26, wherein the composition comprises a branched fatty acid disintegrator.
28. Composition according to paragraph 27, wherein the branched fatty acid disintegrator is selected from the group of sodium isononanoate, isononanoic acid, sodium isooctanoate, isooctanoic acid, sodium neodecanoate, neodecanoic acid, sodium neopentanoate, neopentanoic acid, sodium neoheptanoate, neoheptanoic acid, 3,5,5-trimethylhexanoic acid, 6-methyl-heptanoic acid, 2,2-dimethyloctanoic acid, neopentanoic acid (2,2-dimethylpropanoic acid), 2,2-dimethylpentanoic acid, and salts thereof, or mixtures thereof.
29. Composition according to any of the paragraphs 18-28, wherein the composition is a block or a tablet having a dissolution rate when exposed to 4000 mL of aqueous solution at 68° C. of at least 15 g/minute.
30. Composition according to any of the paragraphs 18-29, wherein the composition further comprises one or more protease inhibitors of which at least one is a peptide aldehyde, a hydrosulfite adduct or a hemiacetal adduct thereof.
31. Composition according to paragraph 30, wherein the protease inhibitor is a peptide aldehyde of the formula P-(A)rL-(B)ₓ—B—H or a hydrosulfite adduct or hemiacetal adduct thereof, wherein:
   i. H is hydrogen;
   ii. B⁰ is a single amino acid residue with L- or D-configuration of the formula —NH—CH(R)—C(═O)—;
   iii. x is 1, 2 or 3 for (B)ₓ, and B is independently a single amino acid connected to B⁰ via the C-terminal of the (B)ₓ amino acid
   iv. L is absent or L is independently a linker group of the formula —C(═O)—, —C(═O)—C(═O)—, —C(═S)—, —C(═S)—C(═S)— or —C(═S)—C(═O)—;
   v. y is 0, 1 or 2 for (A)_{y}, and A is independently a single amino acid residue connected to L via the N-terminal of the (A)_{y} amino acid, with the proviso that if L is absent then A is absent;
   vi. P is selected from the group consisting of hydrogen and an N-terminal protection group, with the proviso that if L is absent then P is an N-terminal protection group;
   vii. R is independently selected from the group consisting of C₁₋₆ alkyl, C₆₋₁₀ aryl or C₇₋₁₀ arylalkyl optionally substituted with one or more, identical or different, substituent's R′;
   viii. R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂; and
   ix. R″ is a C₁₋₆ alkyl group.
32. Composition according to paragraph 31, wherein the hydrosulfite adduct of a peptide aldehyde is of the formula P-(A)_{y}-L-(B)x-N(H)—CHR—CH(OH)—SO₃M, wherein
   i. M is hydrogen or an alkali metal;
   ii. x is 1, 2 or 3 for (B)ₓ, and B is independently a single amino acid connected to B⁰ via the C-terminal of the (B)ₓ amino acid
   iii. L is absent or L is independently a linker group of the formula —C(═O)—, —C(═O)—C(═O)—, —C(═S)—, —C(═S)—C(═S)— or —C(═S)—C(═O)—;
   iv. y is 0, 1 or 2 for (A)_{y}, and A is independently a single amino acid residue connected to L via the N-terminal of the (A)_{y} amino acid, with the proviso that if L is absent then A is absent;
   v. P is selected from the group consisting of hydrogen and an N-terminal protection group, with the proviso that if L is absent then P is an N-terminal protection group;
   vi. R is independently selected from the group consisting of C₁₋₆ alkyl, C₆₋₁₀ aryl or C₇₋₁₀ arylalkyl optionally substituted with one or more, identical or different, substituent's R′;
   vii. R′ is independently selected from the group consisting of halogen, —OH, —OR″, —SH, —SR″, —NH₂, —NHR″, —NR″₂, —CO₂H, —CONH₂, —CONHR″, —CONR″₂, —NHC(═N)NH₂; and
   viii. R″ is a C₁₋₆ alkyl group.
33. Composition according to any of the paragraphs 18-32, wherein the composition comprises an amylase
34. Composition according to paragraph 33, wherein the amylase is an alpha-amylase.
35. Composition according to paragraph 34, wherein the amylase has a sequence identity of at least 70%, such as at least 75%, such as at least 80%, such as at least 85%, such as at least 90%, such as at least 95%, such as at least 98%, such as at least 99%, such as 100%, to the sequence of SEQ ID NO: 1, 2, 3 or 4.
36. Composition according to paragraph 35, wherein the amylase is an alpha-amylase variant comprising a modification in one or more positions corresponding to positions D183*+G184*+R118K+N195F+R320K+R458K or M9L+R118K+G149A+G182T+G186A+D183*+G184*+N195F+M202L+T2571+Y295F+N299Y+R320K+M323T+A339S+E345R+R458K of SEQ ID NO: 1; D183*+G184* or W140Y+D183*+G184*+N195F+V206Y+Y243F+E260G+G304R+G476K of SEQ ID NO: 2; H156Y+A181T+N190F+A209V+Q264S of SEQ ID NO: 3, wherein said alpha-amylase variant has a sequence identity of at least 75% but less than 100% to SEQ ID NO: 1, 2 or 3, respectively, and wherein said alpha-amylase variant has alpha-amylase activity.
37. Composition according to paragraph 35, wherein the amylase is an alpha-amylase variant comprising a modification in one or more positions corresponding to positions H1, N54, V56, K72, G109, F113, R116, T134, W140, W159, W167, Q169, Q172, L173, A174, R181, G182, D183, G184, W189, E194, N195, V206, G255, N260, F262, A265, W284, F289, S304, G305, W347,K391, Q395, W439, W469, R444, F473, G476, and G477 of SEQ ID NO: 4, wherein said alpha-amylase variant has a sequence identity of at least 75% but less than 100% to SEQ ID NO: 4 and wherein said alpha-amylase variant has alpha-amylase activity.
38. Composition according to any of paragraphs 35 and 37, wherein said modification in one or more positions is selected from the group consisting of: H1*, H1A, N54S, V56T, K72R, G109A, F113Q, R116Q, R116H, T134E, W140Y, W140F, W140H, W159Y, W159F, W159H, W167Y, W167H, W167F, Q169E, Q172K, Q172G, Q172N, L173P, A174*, A174S, R181*, G182*, D183*, G184*, G184T, W189Y, W189F, W189H, W189E, W189D, W189Q, W189N, E194D, E194N, E194S, N195F, V206L, V206F, V206Y, G255A, N260G, N260P, N260A, N260G, N260P, N260A, A265G, W284G, W284H, F289H, S304K, S304R, S304Q, S304E, G305K, G305R, G305Q, G305E, W347Y, W347F, W347H, K391A, Q395P, W439N, W439Q, W439T, R444Q, W469T, W469N, F473R, G476R, G476Q, G476E, G476K G477K, G477R, G477Q, and G477E wherein the positions correspond to positions of SEQ ID NO: 4.
39. Composition according to any of paragraphs 35 and 37-38, wherein said at least one alpha-amylase variant comprises a deletion in the positions corresponding to R181+G182; R181+D183; R181+G184; G182+D183; G182+G184; or D183+G184 of SEQ ID NO:4.
40. Composition according to any of paragraphs 35 and 37-39, wherein said alpha-amylase variant in (i) is selected from the group consisting of:
H1*+N54S+V56T+G109A+Q169E+Q172K+A174*+G182*+D183*+N195F+V206L+K391A+G476K;
H1*+N54S+V56T+G109A+R116H+A174S+G182*+D183*+N195F+V206L+K391A+G476K;
H1*+N54S+V56T+K72R+G109A+F113Q+R116Q+W167F+Q172G+A174S+G182*+D183*+G184T+N195F+V206L+K391A+P473R+G476K; H1*+N54S+V56T+G109A+F113Q+R116Q+Q172N+A174S+G182*+D183*+N195F+V206L+A265G+K391A+P473R+G476K;
H1*+N54S+V56T+K72R+G109A+F113Q+W167F+Q172R+A174S+G182*+D183*+N195F+V206L+K391A+G476K;
H1*+N54S+V56T+K72R+G109A+R116H+T134E+W167F+Q172G+L173V+A174S+G182*+D183*+N195F+V206L+G255A+K391A+G476K;
H1*+N54S+V56T+K72R+G109A+R116H+T134E+W167F+Q172G+L173V+A174S+G182*+D183*+N195F+V206L+G255A+K391A+Q395P+T444Q+P473R+G476K;
H1*+N54S+V56T+G109A+T134E+A174S+G182*+D183*+NI195F+V206L+K391A+G476K;
H1*+N54S+V56T+K72R+G109A+A174S+G182*+D183*+N195F+V206L+G255A+K391A+G476K;
H1*+N54S+V56T+K72R+G109A+F113Q+R116Q+W167F+Q172G+A174S+G184T+N195F+V206L+K391A+P473R+G476K, and
H1*+N54S+V56T+G109A+W167F+Q172E+L173P+A174K+G182*+D183*+N195F+V206L+K391A+G476K, of the polypeptide of SEQ ID NO: 4 and wherein said alpha-amylase variant shares at least 80%, such as at least 85%, such as at least 90%, such as at least 93%, such as at least 94%, such as at least 95%, such as at least 96%, such as at least 97%, such as at least 98%, but less than 100% sequence identity with the polypeptide of SEQ ID NO: 4 and wherein said alpha-amylase variant has alpha-amylase activity.
41. Composition according to any of the paragraphs 18-40, wherein the composition comprises a protease.
42. Composition according to paragraph 41, wherein the protease is selected from the group consisting of
   i. a protease having a sequence identity of at least 70%, such as at least 75%, such as at least 80%, such as at least 85%, such as at least 90%, such as at least 95%, such as at least 98%, such as at least 99%, such as 100%, to the sequences of SEQ ID NOs: 5, 6, 7 or 8;
   ii. a protease variant comprising a substitution at one or more positions corresponding to positions 9, 15, 36, 61, 68, 76, 99, 106, 120, 167, 170, 194, 195, 205, 218, 235, 245 or 261 of SEQ ID NO: 3, wherein said protease variant has a sequence identity of at least 75% but less than 100% to SEQ ID NO: 5,
   iii. The protease of SEQ ID NO: 24.
43. Composition according to paragraph 42, wherein the protease is selected from the group consisting of: M222S, *36D+N76D+N120D+G195E+K235L, Y167A+R170S+A194P, S99SE, V68A+S106A, S9R+A15T+V68A+N218D+Q245R, S9R+A15T+G61E+V68A+A194P+V205I+Q245R+N261D and S99AD of SEQ ID NO: 5, wherein said protease variant has a sequence identity of at least 75% but less than 100% to SEQ ID NO: 5 and wherein said protease variant has protease activity.
44. Composition according to any of the paragraphs 18-43, further comprises an additional enzyme selected from the group consisting of an additional protease, lipase, cutinase, an additional amylase, carbohydrase, cellulase, pectinase, mannanase, arabinase, galactanase, xylanase, oxidase, a licheninase, a laccase and/or peroxidase.
45. Composition according to paragraph 44, wherein the licheninase is a polypeptide having at least 89% sequence identity to the mature polypeptide of the sequence selected from the group consisting of: SEQ ID NO: 18, SEQ ID NO: 19, SEQ ID NO: 20, SEQ ID NO: 21 or SEQ ID NO: 22.
46. Composition according to paragraph 45, wherein the licheninase is a His-tagged recombinant mature polypeptide of SEQ ID NO: 18, SEQ ID NO: 19, SEQ ID NO: 20, SEQ ID NO: 21 or SEQ ID NO: 22.
47. Composition according to paragraph 44, wherein the licheninase is SEQ ID NO: 23.
48. A method for removing and/or reducing soil on a surface, wherein a cleaning cycle comprises the steps of:
   i. A washing step, wherein the surface is exposed to a wash liquor comprising
      a. an amylase and/or a protease and optionally detergent components, or
      b. a warewashing detergent composition according to paragraphs 18-47,
   ii. Optionally draining part of the wash liquor,
   iii. Optionally rinsing the surface,
   iv. Optionally drying the surface;
wherein the surface is exposed to the wash liquor for a time period in the range of 10 to 240 seconds.
49. Method according to paragraph 48, wherein cleaning cycle comprises a rinsing step.
50. Method according to any of the paragraphs 48-49, wherein cleaning cycle comprises a drying step.
51. Method according to any of the paragraphs 48-50, wherein cleaning cycle comprises a rinsing and a drying step.
52. Method according to any of the paragraphs 48-51, wherein clean water is used for the rinsing step optionally together with a rinse aid.
53. Method according to any of the paragraphs 48-52, wherein the method comprises a soaking step before step a.
54. Method according to any of the paragraphs 48-53, wherein the method comprises a draining step, wherein part of the wash liquor is drained.
55. Method according to paragraph 54, wherein clean water from rinsing step c replaces the wash liquor drained.
56. Method according to paragraph 54-55, wherein 5-15% of the wash liquor is replaced by clean water.
57. Method according to paragraph 56, wherein 7.5% of the wash liquor is replaced by clean water.
58. Method according to any of the paragraphs 48-57, wherein the wash liquor is re-used in a subsequent cleaning cycle.
59. Method according to any of the paragraphs 48-58, wherein the surface undergoes the cleaning cycle 1 time.
60. Method according to any of the paragraphs 48-59, wherein the surface undergoes the cleaning cycle 2 times, 3 times, 4 times or 5 times.
61. Method according to any of the paragraphs 48-60, wherein the wash liquor comprises a composition according to any of the paragraphs 18-47.
62. Method according to any of the paragraphs 48-61, wherein the wash liquor is supplied with enzymes and the detergent components from separate containers.
63. Method according to paragraph 62, wherein wash liquor is supplied with amylase and/or protease and optionally additional enzymes from separate containers.
64. Method according to any of the paragraphs 48-63, wherein the surface is exposed to the amylase and/or protease for a time period of 10 to 220 seconds, such in the range of 10 to 200 seconds, in the range of 10 to 180 seconds, in the range of 10 to 160 seconds, in the range of 10 to 140 seconds, in the range of 10 to 120 seconds, in the range of 10 to 100 seconds, in the range of 10 to 80 seconds, in the range of 10 to 70 seconds, in the range of 10 to 66 seconds, in the range of 20 to 66 seconds, in the range of 25 to 66 seconds, in the range of 28 to 66 seconds, in the range of 28 to 60 seconds, in the range of 28 to 55 seconds, in the range of 28 to 50 seconds or in the range of 28 to 45 seconds.
65. Method according to any of the paragraphs 48-64, wherein the pH of the wash liquor is in the range of 7-10.5.
66. Method according to paragraph 65, wherein the pH is in the range of 7.5-10.5, such as in the range of 7.5-10, in the range of 7.5-9.5, in the range of 7.5-9.0, in the range of 7.5-8.5, in the range of 7.5-8.2, or in the range of 7.8-8.2.
67. Method according to any of the paragraphs 48-66, wherein the temperature of the wash liquor is in the range of 50-95° C.
68. Method according to paragraph 67, wherein the temperature is in the range of 50-90° C., in the range of 50-85° C., in the range of 50-80° C., in the range of 50-75° C., in the range of 50-70° C., in the range of 50-65° C., 55-62° C., such as in the range of 58-62° C.
69. Method according to any of the paragraphs 48-68, wherein the method is carried out in a warewashing machine selected from the group consisting of a door warewashing machine, a hood warewashing machine, a conveyor warewashing machine, an undercounter warewashing machine, a glasswasher, a flight warewashing machine, a pot and pan warewashing machine and a utensil washer.
70. Method according to any of the paragraphs 48-69, wherein the surface is the inner surface of a warewashing machine or the surface of a ware.
71. Method according to any of the paragraphs 48-70, wherein the concentration of the detergent composition is in the range of 0.5-5 g/liter wash liquor.
72. Method according to paragraph 71, wherein the concentration is in the range of 1-4 g/liter wash liquor, such as in the range of 1.5-3 g/liter wash liquor.
73. Method according to paragraph 72, wherein the concentration is 2 g/liter wash liquor.

### EXAMPLES

### Warewashing Detergent Compositions

| Type | Producer | Brand | pH | Component | wt. % |
|---|---|---|---|---|---|
| Liquid | Sealed Air | Suma | >12 | Sodium hydroxide | 10-20 |
| | (Diversey) | Ultra Pur- | | | |
| | | Eco | | | |
| Solid | Ecolab | Apex | 10.5-11.4 (1%) | Sodium carbonate | 50-100 |
| | | Power | | HEDP chelator | 5-10 |
| | | | | Alcohol ethoxylate | 2.5-3 |
| | | | | Sodium dichloroisocyanurate | 1-2.5 |
| | | | | (disinfectant) | |
| Solid | Ecolab | Apex | 10.5-11.5 (1%) | Sodium carbonate | >50 |
| | | Ultra | | troclosene sodium, dihydrate | 1-5 |
| | | | | Alcohol ethoxylate | 1-5 |
| | | | | oxirane, methyl-, polymer with | 1-5 |
| | | | | oxirane | |
| | | | | Sodium Metasilicate | 1-5 |
| Liquid | Spartan | Sparclean | 13.5-14.0 | water | 40-70 |
| | Chemical | All | | sodium hydroxide | 10-30 |
| | Company | Temperature | | potassium hydroxide | 7-13 |
| | | Detergent | | MGDA chelator | 3-7 |
| | | [50] | | detergent polymer | 1-5 |
| Liquid | Novadan | Bistro 741 | 12.5 (1%) | Sodium hydroxide | 5-15 |
| | | (Økoren | | 1.2-phosphonobutane-1,2,4- | 1-5 |
| | | Bistro) | | tricarboxylic acid | |
| | | | | MGDA chelator | 1-5 |
| | | | | Phosphonates | <5 |
| | | | | Polycarboxylates | <5 |
| Liquid | Integra | VISION - | 12.6 (1%) | Sodium hydroxide | 13 |
| | Cleaning | Environmentally | | | |
| | solutions | preferable | | | |
| | | warewash | | | |
| | | detergent | | | |
| Liquid | Integra | CONQUER - | 12.6 (1%) | Sodium hydroxide | 15.6 |
| | Cleaning | Heavy | | Sodium hypochlorite | 3 |
| | solutions | duty | | | |
| | | warewash | | | |
| | | detergent | | | |
| Liquid | Integra | ECLIPSE - | 12.5 (1%) | EDTA | 16.2 |
| | Cleaning | All-temp | | Sodium hydroxide | 13 |
| | solutions | warewash | | | |
| | | detergent | | | |
| Liquid | Integra | PROTECT - | >13 | Sodium hydroxide | 6.5 |
| | Cleaning | Warewashing | | Sodium hypochlorite | 2.6 |
| | solutions | detergent | | | |
| | | w/corrosion | | | |
| | | inhibitors | | | |
| Solid | Sunburst | Encore - | 10-11.5 | Sodium Metasilicate | 30-50 |
| | Chemicals | Pan- | | Pentasodium Triphosphate | 10-30 |
| | | Washing | | Sodium Hydroxide | 5-20 |
| | | Detergent | | 2-Propenoic acid, telomer with | 3-10 |
| | | | | sodium hydrogen sulfite, sodium | |
| | | | | salt | |
| Solid | Sunburst | Applause - | 11.0-11.5 | Sodium Hydroxide | 20-40 |
| | Chemicals | Dishwasher | | Pentasodium Triphosphate | 20-36 |
| | | Detergent | | Sodium Silicate | 20-30 |
| | | | | 2-Propenoic acid, telomer with | 3-10 |
| | | | | sodium hydrogen sulfite, sodium | |
| | | | | salt | |
| Powder | Sunburst | Concert - | 11.0-11.8 | Sodium Carbonate | 30-50 |
| | Chemicals | Dishwasher | | Sodium Hydroxide | 30-40 |
| | | Detergent | | Pentasodium Triphosphate | 10-30 |
| | | | | Sodium Dichloroisocyanurate | 2-4 |
| Solid | Sunburst | Solid | 10.8-11.8 | Sodium Hydroxide | 30-50 |
| | Chemicals | Performance - | | Pentasodium Triphosphate | 20-40 |
| | | Dishwasher | | 2-propenoic acid, homopolymer, | 3-10 |
| | | Detergent | | sodium salt | |
| | | | | Sodium Carbonate | 1-5 |
| Solid | Sunburst | Solid | <11.5 | Sodium Hydroxide | 30-50 |
| | Chemicals | Green 99 | | Sodium Carbonate | 10-20 |
| | | Dish | | Water | 5-15 |
| | | Machine | | Polyacrylic Acid | 3-10 |
| | | Detergent - | | Methyl-oxirane polymer with | 0.1-3 |
| | | Dishwasher | | oxirane | |
| | | Detergent | | | |
| Liquid | Sunburst | Result | 12.5-13.75 | Sodium Hydroxide | 10-30 |
| | Chemicals | 100- | | 2-Propenoic acid, telomer with | 3-10 |
| | | Glasswasher | | sodium hydrogen sulfite, sodium | |
| | | Detergent | | salt | |
| | | | | EDTA | 1-5 |
| Liquid | Sunburst | Result | 11.0-12.0 | Potassium Hydroxide | 10-30 |
| | Chemicals | Shield D - | | Sodium Silicate | 10-30 |
| | | Dishwasher | | Tetrapotassium Pyrophosphate | 5-10 |
| | | Detergent | | 2-Propenoic acid, telomer with | 2-8 |
| | | | | sodium hydrogen sulfite, sodium | |
| | | | | salt | |
| Liquid | Sunburst | Ultima | 12.0-13.5 | Sodium Hydroxide | 20-40 |
| | Chemicals | Result - | | 2-Propenoic acid, telomer with | 5-20 |
| | | Dishwasher | | sodium hydrogen sulfite, sodium | |
| | | Detergent | | salt | |
| Liquid | U S | ALL | >13 | Potassium Hydroxide | 17 |
| | Chemical | TEMP | | Sodium Hydroxide | 2 |
| | | H.D. | | | |
| Liquid | U S | Buffered | >13 | Potassium Hydroxide | 10.9 |
| | Chemical | warewash | | Sodium Silicate | 10.3 |
| | | detergent | | Sodium hypochlorite | 2.2 |
| Liquid | Dr. Weigert | Neodisher | 9.4-9.6 | <5% phosphonates, | |
| | | Bio Clean | | polycaboxylates | |

### Warewashing Detergent Composition

| Component | Composition (wt. %) |
|---|---|
| Distilled water | 30-70 |
| Sodium carbonate (pH regulator) | 2-10 |
| Sodium bicarbonate (pH regulator) | 2-10 |
| 1,2-propylene glycol (Solvent) | 10-20 |
| Sodium xylene sulfonate 40% (Solubilizer) | 5-20 |
| Dissolvine GL38 (Chelator) | 2-10 |
| Bio-Soft N91-2.5 (Non-ionic surfactant) | 1-5 |

### Rinse Aid Compositions

| Type | Producer | Brand | pH | Component | wt. % |
|---|---|---|---|---|---|
| Liquid | US | Auto-Dri | 7 (conc.) | SODIUM | 20 |
| | Chemical | | | XYLENESULFONATE | |
| | | | | ISOPROPYL ALCOHOL | 5 |
| Liquid | US | Dri-Fast | 5 (conc.) | AMINE POLYGLYCOL | 6.9 |
| | Chemical | | | CONDENSATE | |
| | | | | ALCOHOL ALKOXYLATE | 3 |
| Liquid | US | Drop-Off | 5 (conc.) | AMINE POLYGLYCOL | 11.8 |
| | Chemical | | | CONDENSATE | |
| | | | | ALCOHOL ALKOXYLATE | 4.8 |
| Liquid | US | H-D-R | Below 2.5 | HYDROXYACETIC ACID | 14 |
| | Chemical | | | | |
| | | | | ALCOHOL ALKOXYLATE | 20 |
| Liquid | US | LOW ENERGY RINSE AID | 7.5 | POLYOXYPROPYLENE- | 10 |
| | Chemical | | | POLYOXYETHYLENE | |
| Liquid | US | MICROTECH SURETY | 2.5 | SODIUM | 20 |
| | Chemical | PREMIUM RINSE ADDITIVE | | XYLENESULFONATE | |
| | | | | CITRIC ACID | 8 |
| Liquid | US | RINSE AID E.S. | 5.0 | AMINE POLYGLYCOL | 7.4 |
| | Chemical | | | CONDENSATE | |
| | | | | ALCOHOL ALKOXYLATE | 3.2 |
| Liquid | US | RINSE-RITE | 5.0 | AMINE POLYGLYCOL | 7.8 |
| | Chemical | | | CONDENSATE | |
| | | | | ALCOHOL ALKOXYLATE | 4.5 |
| Solid | Sunburst | Clarity | 7-9 | Alcohol Alkoxylate | 70-90 |
| | Chemicals | | | Urea | 10-30 |
| Liquid | Sunburst | Sparkle | 7-10 | Alcohol Alkoxylate | 20-40 |
| | Chemicals | | | | |
| Liquid | Sunburst | TD 360 | 4-5 | Alcohol Alkoxylate | 10-20 |
| | Chemicals | | | Acrylic acid | 10-20 |
| Liquid | Sunburst | Ultima Sparkle | 4-5 | Alkoxylated Alcohol | 10-50 |
| | Chemicals | | | 2-propenoic acid telemer, | 3-10 |
| | | | | sodium salt | |
| Solid | Sunburst | Solid Green 23 Rinse Aid | 8-9 (diluted) | Methyl-oxirane polymer with | 60-80 |
| | Chemicals | | | oxirane | |
| | | | | Urea | 10-30 |
| | | | | Sodium Xylene Sulfonate | 1-10 |
| | | | | Triarylmethane | <0.01 |
| Solid | Ecolab | APEX RINSE ADDITIVE TSC | 7.4 | oxirane, methyl-, polymer | 30-60 |
| | | | | with oxirane | |
| | | | | Urea | 10-30 |
| | | | | alcohols, c10-16, ethoxylated | 10-30 |
| | | | | Fattyalcohol ethoxylates > | 5-10 |
| | | | | 5EO | |
| Liquid | Sealed Air | Suma Rinse A5 Rinse Aid | 8 | Sodium alkyl sarcosinate | >0.1%-<1% |
| | (Diversey) | | | | |

### Assays

### Assay I

Items are immersed in an iodine solution (0.025 M) for approximately 30 seconds. The iodine binds to soil on the item and thereby makes the soil on the item visible by the naked eye. The items, either clean or tested under the same conditions, are evaluated by a trained test person on a scale from 1 to 10. 1 is assigned to items on which most soil is visible (most dirty) and 10 is assigned to items on which less soil invisible (most clean).
Assay II—Alpha-Amylase Activity Assay—pNP-G7 Assay

The alpha-amylase activity may be determined by a method employing the G7-pNP substrate. G7-pNP which is an abbreviation for 4,6-ethylidene(G₇)-p-nitrophenyl(Gi)-α,D-maltoheptaoside, a blocked oligosaccharide which can be cleaved by an endo-amylase, such as an alpha-amylase. Following the cleavage, the alpha-Glucosidase included in the kit digest the hydrolysed substrate further to liberate a free PNP molecule which has a yellow color and thus can be measured by visible spectophometry at λ=405 nm (400-420 nm.). Kits containing G7-pNP substrate and alpha-Glucosidase is manufactured by Roche/Hitachi (cat. No. 11876473).

Reagents:

The G7-pNP substrate from this kit contains 22 mM 4,6-ethylidene- G7-pNP and 52.4 mM HEPES (2-[4-(2-hydroxyethyl)-1-piperazinyl]-ethanesulfonic acid), pH 7.0).

The alpha-Glucosidase reagent contains 52.4 mM HEPES, 87 mM NaCl, 12.6 mM MgCl₂, 0.075 mM CaCl₂, ≥4 kU/L alpha-glucosidase).

The substrate working solution is made by mixing 1 mL of the alpha-Glucosidase reagent with 0.2 mL of the G7-pNP substrate. This substrate working solution is made immediately before use.

Dilution buffer: 50 mM MOPS, 0.05% (w/v) Triton X100 (polyethylene glycol p-(1,1,3,3-tetramethylbutyl)-phenyl ether (C₁₄H₂₂O(C₂H₄O)ₙ (n=9-10))), 1 mM CaCl₂), pH8.0.

Procedure:

The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 sec. over 5 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### Assay III—Alpha-Amylase Activity Assay—Phadebas Activity Assay

The alpha-amylase activity may also be determined by a method using the Phadebas substrate (from for example Magle Life Sciences, Lund, Sweden). A Phadebas tablet includes interlinked starch polymers that are in the form of globular microspheres that are insoluble in water. A blue dye is covalently bound to these microspheres. The interlinked starch polymers in the microsphere are degraded at a speed that is proportional to the alpha-amylase activity. When the alpha-amylase degrades the starch polymers, the released blue dye is water soluble and concentration of dye can be determined by measuring absorbance at 620 nm. The concentration of blue is proportional to the alpha-amylase activity in the sample.

The alpha-amylase sample to be analyzed is diluted in activity buffer with the desired pH. Two substrate tablets are suspended in 5 mL activity buffer and mixed on magnetic stirrer. During mixing of substrate transfer 150 μl to microtiter plate (MTP) or PCR-MTP. Add 30 μl diluted amylase sample to 150 μl substrate and mix. Incubate for 15 minutes at 37° C. The reaction is stopped by adding 30 μl 1M NaOH and mix. Centrifuge MTP for 5 minutes at 4000×g. Transfer 100 μl to new MTP and measure absorbance at 620 nm.

The alpha-amylase sample should be diluted so that the absorbance at 620 nm is between 0 and 2.2, and is within the linear range of the activity assay.

### Assay IV—Alpha-Amylase Activity Assay—Amylazyme Activity Assay

The alpha-amylase activity may also be determined by a method using the Amylazyme substrate (Megazyme® Amylazyme Test, supplied by Megazyme for the assay of cereal and bacterial amylases) comprising AZCL-amylose, which has been mixed with lactose and magnesium stearate and tabletted. A blue dye is covalently bound to these microspheres. The interlinked amylose polymers in the microsphere are degraded at a speed that is proportional to the alpha-amylase activity. When the alpha-amylase degrades the starch polymers, the released blue dye is water soluble and concentration of dye may be determined by measuring absorbance at 590 nm. The concentration of blue is proportional to the alpha-amylase activity in the sample.

The alpha-amylase sample to be analyzed is diluted in activity buffer with the desired pH. Two substrate tablets are suspended in 5 mL activity buffer and mixed on magnetic stirrer. During mixing of substrate 150 μl is transferred to a microtiter plate (MTP) or PCR-MTP. Next, 25 μl diluted amylase sample is added to 150 μl substrate and mixed. The mixture is incubated for 10 minutes at 37° C. The reaction is stopped by adding 25 μl 1M NaOH and mixed. MTP is centrifuged for 5 minutes at 4000×g, followed by transferring 100 μl to a new MTP and absorbance is measured at 590 nm.

### Assay V—Protease Activity Assays:

1) Suc-AAPF-pNA Activity Assay:

The proteolytic activity can be determined by a method employing the Suc-AAPF-PNA substrate. Suc-AAPF-PNA is an abbreviation for N-Succinyl-Alanine-Alanine-Proline-Phenylalanine-p-Nitroanilide, and it is a blocked peptide which can be cleaved by endo-proteases. Following cleavage a free PNA molecule is liberated and it has a yellow colour and thus can be measured by visible spectrophotometry at wavelength 405 nm. The Suc-AAPF-PNA substrate is manufactured by Bachem (cat. no. L1400, dissolved in DMSO).

The protease sample to be analyzed was diluted in residual activity buffer (100 mM Tris pH8.6). The assay was performed by transferring 60 μl of diluted enzyme samples to 96 well microtiter plate and adding 140 μl substrate working solution (0.72 mg/ml in 100 mM Tris pH8.6). The solution was mixed at room temperature and absorption is measured every 20 sec. over 5 minutes at OD 405 nm.

The slope (absorbance per minute) of the time dependent absorption-curve is directly proportional to the specific activity (activity per mg enzyme) of the protease in question under the given set of conditions. The protease sample should be diluted to a level where the slope is linear.

### Assay VI—Dissolving Rate Test Procedure

The test procedures used in the current invention include three developed test procedures. The first test procedure is a dissolving rate test procedure. This test procedure measures the dissolution rate of the solid when it is added to water at various temperatures. The test procedure is as follows:
1. Bring 3500 mIs of soft water to designate temperature in a 4000 ml beaker on a hotplate.
2. Add screen support to beaker (screen support positions sample 7.5 cm from bottom of beaker).
3. Record weight solid sample to be tested.
4. When water reaches designated temperature, add sample and start stopwatch.
5. Record time when no sample remains on the screen.
All dissolving rate test results presented below were performed according to the above procedure at 155[deg.] F. unless otherwise noted. The dissolving rate test procedure may also be performed at other designated temperatures at or above room temperature and below boiling point of the aqueous solution. Example designate temperatures include, for example, but are not limited to 130[deg.] F. and 190[deg.] F.
Standard room temperature, pressure, etc. conditions are otherwise applicable.

### Example 1

### Warewash Trials on Plates

Oat porridge soil is prepared by adding 50 gram of porridge oats to 250 ml of UHT (ultra-high-temperature processed) cow milk (homogenized, 1.5% fat) and 750 ml of synthetized water (16.8° dH), heating steadily while stirring continuously and boil for 10 minutes.
Using a brush, 3 g of hot oat porridge is spread evenly on the inner plate surface. The rim of the plates is kept free. Diameter of inner plate is 15 cm. The soiled plates are dried for 2 hours at 80° C. in a thermal cabinet. When the plates have cooled to room temperature, they are washed.
Plates pre-soiled with oat porridge were washed during 1 cleaning cycle in a Hobart AUXX hooded warewasher operating on a 90 second cleaning cycle comprising a washing step (66 seconds (s), 60-65° C.), draining of 2.5 I1 wash liquor (15 s), rinsing with 2.5 I1 clean water (9 s, 85° C.) and a drying step (31 s). Water hardness of the wash liquor was 0° dH. In order to simulate the conditions in a real life kitchen, a ballast soil is added to the cleaning cycle of the warewasher. 1 g/L wash liquor ballast soil was added to the sump before starting the cleaning cycle.
The cleaning performance of four different setups were tested using a warewash model detergent composition (A) without addition of enzymes, (B) with addition of protease, (C) with addition of amylase and (D) with addition of both protease and amylase. Ten plates were tested for each setup. To eliminate the risks of transferring organic materials between each setup the warewasher is purged (turned off) and restarted after each cleaning cycle. During this process the inner surface of the warewasher is cleaned.
The detergent composition used in the wash trials was made as defined in Table 1. The detergent composition was pH neutral, i.e. enzyme friendly and added to the warewasher at 2 g/L wash liquor. In the wash trials the enzymes (see Table 2) were added to the warewasher as shown.

TABLE 1
Chemical composition of the warewash detergent
(named NZ detergent in figure) without enzymes

| Component | Composition (wt. %) |
|---|---|
| Distilled water | 56.00 |
| Sodium carbonate (pH regulator) | 2.18 |
| Sodium bicarbonate (pH regulator) | 3.62 |
| 1,2-propylene glycol (Solvent) | 15.60 |
| Sodium xylene sulfonate 40% (Solubilizer) | 12.00 |
| Dissolvine GL38 (Chelator) | 8.60 |
| Bio-Soft N91-2.5 (Non-ionic surfactant) | 2.00 |

TABLE 2
Enzymes used in the wash trials

| Enzymes | Concentration used in wash trials |
|---|---|
| Amylase: | 0.286 mg enzyme protein/g |
| SEQ ID NO: 4 + H1* + N54S + V56T + K72R + G109A + | wash liquor |
| F113Q + R116Q + W167F + Q172G + A174S + G182* + | |
| D183* + G184T + N195F + V206L + K391A + P473R + | |
| G476K | |
| Protease: | 0.446 mg enzyme protein/g |
| SEQ ID NO: 24 | wash liquor |

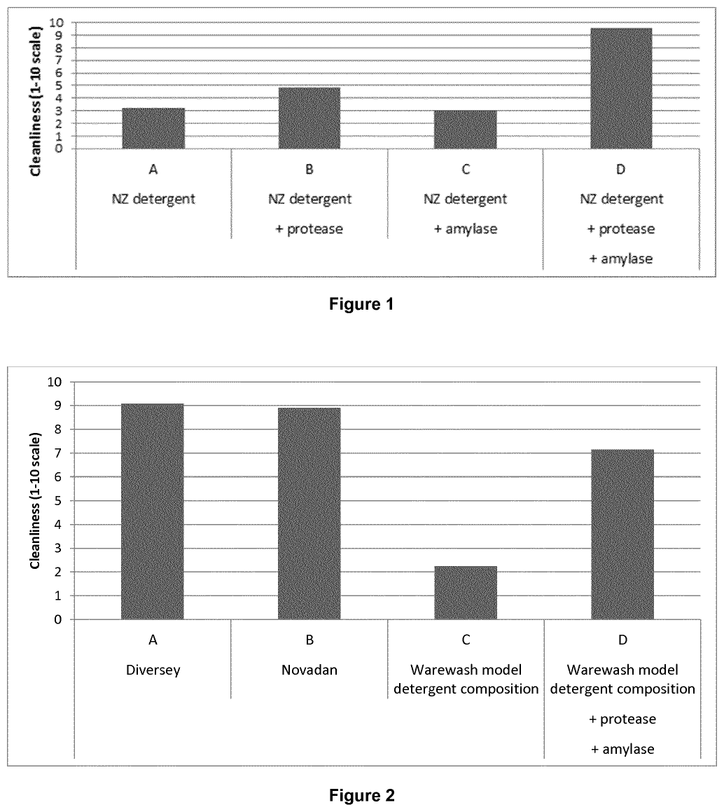

The protease of table 2 is used together with protease inhibitor Cbz-Gly-Ala-Tyr-H.
Once the plates were washed, the performance of the wash is evaluated trained test persons according to Assay I. The result of the evaluation is shown in tables 3-6 below and in FIG. 1.
FIG. 1: A test panel consisting of 10 trained test persons has been evaluating the cleanliness of all plates relative to each other (Assay I). This figure shows that a significant performance improvement was present when an enzyme mixture of the Amylase and the Protease of Table 2 was added to the warewash detergent (Table 1). When the plates were washed using either amylase or protease, the cleaning performance was not significant. This indicates that a high degree of amylase/protease synergy is present.

TABLE 3
Treatment A: a warewash model detergent composition

| Plate | Test person Number | | | | | | | | | |
|---|---|---|---|---|---|---|---|---|---|---|
| Number | 1 | 2 | 3 | 4 | 5 | 6 | 7 | 8 | 9 | 10 |
| 1 | 3 | 3 | 3 | 6 | 3 | 5 | 7 | 5 | 8 | 5 |
| 2 | 2 | 2 | 2 | 6 | 2 | 3 | 5 | 4 | 8 | 4 |
| 3 | 2 | 2 | 2 | 5 | 2 | 4 | 5 | 4 | 5 | 6 |
| 4 | 2 | 2 | 2 | 5 | 3 | 3 | 4 | 4 | 5 | 4 |
| 5 | 2 | 2 | 2 | 4 | 2 | 3 | 4 | 4 | 6 | 6 |
| 6 | 2 | 3 | 2 | 5 | 3 | 3 | 5 | 4 | 6 | 4 |
| 7 | 1 | 1 | 1 | 1 | 1 | 2 | 1 | 2 | 1 | 2 |
| 8 | 1 | 1 | 1 | 3 | 2 | 2 | 3 | 4 | 2 | 3 |
| 9 | 2 | 2 | 2 | 4 | 3 | 4 | 5 | 4 | 7 | 7 |
| 10 | 1 | 1 | 1 | 1 | 2 | 1 | 1 | 4 | 1 | 3 |

TABLE 4
Treatment B: a warewash model detergent
composition with addition of protease

| Plate | Test person Number | | | | | | | | | |
|---|---|---|---|---|---|---|---|---|---|---|
| Number | 1 | 2 | 3 | 4 | 5 | 6 | 7 | 8 | 9 | 10 |
| 1 | 1 | 2 | 2 | 4 | 3 | 2 | 5 | 4 | 3 | 3 |
| 2 | 2 | 3 | 3 | 6 | 5 | 5 | 7 | 5 | 6 | 7 |
| 3 | 2 | 3 | 3 | 5 | 5 | 5 | 7 | 5 | 5 | 7 |
| 4 | 3 | 4 | 3 | 6 | 4 | 6 | 7 | 5 | 7 | 7 |
| 5 | 2 | 3 | 2 | 4 | 4 | 3 | 5 | 4 | 4 | 4 |
| 6 | 1 | 2 | 2 | 4 | 3 | 3 | 5 | 4 | 3 | 3 |
| 7 | 4 | 6 | 4 | 7 | 8 | 8 | 9 | 7 | 8 | 8 |
| 8 | 2 | 2 | 3 | 5 | 4 | 4 | 6 | 5 | 5 | 5 |
| 9 | 3 | 5 | 3 | 7 | 7 | 7 | 8 | 6 | 8 | 8 |
| 10 | 4 | 5 | 3 | 7 | 8 | 9 | 8 | 7 | 8 | 8 |

TABLE 5
Treatment C: a warewash model detergent
composition with addition of amylase

| Plate | Test person Number | | | | | | | | | |
|---|---|---|---|---|---|---|---|---|---|---|
| Number | 1 | 2 | 3 | 4 | 5 | 6 | 7 | 8 | 9 | 10 |
| 1 | 1 | 1 | 2 | 2 | 1 | 2 | 2 | 3 | 1 | 1 |
| 2 | 2 | 2 | 3 | 4 | 3 | 3 | 5 | 5 | 7 | 3 |
| 3 | 2 | 3 | 3 | 6 | 5 | 5 | 7 | 5 | 7 | 6 |
| 4 | 2 | 2 | 3 | 5 | 5 | 3 | 5 | 5 | 6 | 3 |
| 5 | 2 | 2 | 3 | 5 | 3 | 4 | 5 | 5 | 6 | 3 |
| 6 | 1 | 1 | 2 | 2 | 1 | 1 | 2 | 2 | 1 | 1 |
| 7 | 2 | 2 | 2 | 4 | 3 | 4 | 4 | 5 | 6 | 6 |
| 8 | 1 | 1 | 2 | 2 | 1 | 2 | 1 | 2 | 1 | 1 |
| 9 | 2 | 2 | 3 | 5 | 5 | 4 | 5 | 5 | 7 | 6 |
| 10 | 1 | 1 | 2 | 3 | 1 | 2 | 2 | 4 | 2 | 2 |

TABLE 6
Treatment D: a warewash model detergent composition
with addition of both protease and amylase

| Plate | Test person Number | | | | | | | | | |
|---|---|---|---|---|---|---|---|---|---|---|
| Number | 1 | 2 | 3 | 4 | 5 | 6 | 7 | 8 | 9 | 10 |
| 1 | 10 | 9 | 10 | 9 | 10 | 9 | 10 | 9 | 9 | 9 |
| 2 | 10 | 10 | 10 | 10 | 10 | 10 | 10 | 10 | 10 | 10 |
| 3 | 10 | 10 | 10 | 10 | 10 | 10 | 10 | 10 | 10 | 10 |
| 4 | 10 | 10 | 10 | 10 | 10 | 10 | 10 | 10 | 10 | 10 |
| 5 | 10 | 9 | 10 | 9 | 10 | 9 | 10 | 9 | 9 | 10 |
| 6 | 10 | 9 | 10 | 9 | 9 | 10 | 10 | 9 | 10 | 9 |
| 7 | 10 | 9 | 10 | 10 | 10 | 10 | 10 | 10 | 10 | 10 |
| 8 | 8 | 8 | 9 | 7 | 8 | 8 | 9 | 8 | 8 | 8 |
| 9 | 10 | 9 | 10 | 9 | 10 | 9 | 10 | 9 | 9 | 9 |
| 10 | 10 | 9 | 10 | 9 | 10 | 9 | 10 | 9 | 9 | 9 |

### Example 2

### Warewash Trials on Plates

Oat porridge soil is prepared by adding 50 gram of porridge oats to 250 ml of UHT (ultra-high-temperature processed) cow milk (homogenized, 1.5% fat) and 750 ml of synthetized water (16.8° dH), heating steadily while stirring continuously and boil for 10 minutes.
Using a brush, 3 g of hot oat porridge is spread evenly on the inner plate surface. The rim of the plates is kept free. Diameter of inner plate is 15 cm. The soiled plates are dried for 2 hours at 80° C. in a thermal cabinet. When the plates have cooled to room temperature, they are washed.
Plates pre-soiled with oat porridge were washed during 1 cleaning cycle in a Hobart AUXX hooded warewasher operating on a 90 second cleaning cycle comprising a washing step (66 seconds (s), 60-65° C.), draining of 2.5 I1 wash liquor (15 s), rinsing with 2.5 I1 clean water (9 s, 85° C.) and a drying step (31 s). Water hardness of the wash liquor was 0° dH. In order to simulate the conditions in a real life kitchen, a ballast soil is added to the cleaning cycle of the warewasher. 1 g/L wash liquor ballast soil was added to the sump before starting the cleaning cycle.
The cleaning performance of four different setups were tested using (A) Diversey Suma Ultra Pur-Eco L2 a commercial high pH warewash detergent without enzymes (B) a different commercial high pH warewash detergent without enzymes Novadan Bistro 741, (C) a mild pH model detergent composition without addition of enzymes and (D) the model detergent composition with addition of protease and amylase. Ten plates were tested for each setup. To eliminate the risks of transferring organic materials between each setup the warewasher is purged (turned off) and restarted after each cleaning cycle. During this process the inner surface of the warewasher is cleaned.
The detergent composition used in the wash trials was made as defined in Table 1. The detergent composition was pH neutral, i.e. enzyme friendly and added to the warewasher at 2 g/L wash liquor. In the wash trials the enzymes (see Table 2) were added to the warewasher as shown.
The chemical composition of the warewashing detergent composition, the enzymes used and concentration of enzymes are the same as in example 1 and shown in tables 1 and 2.
Once the plates were washed, the performance of the wash is evaluated by trained test persons according to Assay I. The result of the evaluation is shown in table 7 below. A test panel consisting of two trained test persons has been evaluating the cleanliness of all plates relative to each other (Assay I). FIG. 2 shows the average of the data in table 10. It can be seen that a significant improvement in wash performance was present when an enzyme mixture of the Amylase and the Protease of Table 2 was added to the mild pH detergent (Table 1) compared to the wash performance of the mild pH detergent (Table 1) without enzymes. table 7 below. Iso show a significant wash performance loss when using the mild pH detergent (table 1) compared to conventional high pH commercial warewash detergents. However, it was seen that it is possible to reduce the cleaning performance gab by adding protease and amylase (Table 2) to the mild pH detergent (Table 1).

TABLE 7
A test panel consisting of two trained test persons has been evaluating
the cleanliness of all plates relative to each other (Assay I).

| Plate | Treatment A | | Treatment B | | Treatment C | | Treatment D | |
|---|---|---|---|---|---|---|---|---|
| number | 1 | 2 | 1 | 2 | 1 | 2 | 1 | 2 |
| 1 | 9 | 9 | 9 | 7 | 1 | 3 | 8 | 7 |
| 2 | 10 | 10 | 10 | 9 | 2 | 4 | 9 | 7 |
| 3 | 10 | 10 | 9 | 9 | 2 | 5 | 9 | 7 |
| 4 | 9 | 9 | 9 | 9 | 2 | 5 | 7 | 7 |
| 5 | 9 | 9 | 10 | 9 | 2 | 4 | 7 | 7 |
| 6 | 9 | 8 | 9 | 8 | 1 | 2 | 6 | 6 |
| 7 | 9 | 8 | 9 | 8 | 1 | 3 | 7 | 7 |
| 8 | 9 | 9 | 9 | 8 | 1 | 2 | 6 | 6 |
| 9 | 9 | 9 | 10 | 8 | 1 | 1 | 8 | 7 |
| 10 | 9 | 9 | 10 | 9 | 1 | 2 | 8 | 7 |

### Example 3

### Warewash Trials on Plates

Oat porridge soil and pre-soiled plates are prepared as described in example 1, first paragraph with the modification that after the oat porridge have boiled for 10 minutes the oat porridge is thoroughly blended for approximately 5 minutes using a hand blender.

The presoiled plates were washed during 1 cleaning cycle in a Hobart AUXX hooded warewasher at same washing program as described in example 1, second paragraph.

The cleaning performance of six different treatments were tested using a commercial mild in-wash pH warewash detergent (A) without addition of enzymes, (B) with addition of amylase and protease, (C) with addition of amylase, protease and licheninase, (D) with addition of licheninase only, (E) with addition of amylase only and (F) with addition of amylase and licheninase. Six plates were tested for each treatment. To eliminate the risks of transferring organic materials between each setup the warewasher is purged (turned off) and restarted after each cleaning cycle. During this process the inner surface of the warewasher is cleaned with tap water.

The commercial warewashing detergent composition used, Dr. Weigert, neodisher BioClean, is a liquid concentrate free of phosphates and free of active chlorine. The formulation is regarded as non-hazardous, non-corrosive and non-irritant. In present experiment the detergent composition was added to the warewasher at 2 g/L wash liquor and the application pH was neutral, i.e. enzyme friendly. In the wash trials the enzymes (see Table ) were added to the warewasher as shown.

TABLE 8
Enzymes used in the wash trials

| Enzymes | Concentration used in wash trials |
|---|---|
| Amylase: | 0.286 mg enzyme protein/g wash |
| SEQ ID NO: 4 + H1* + N54S + V56T + K72R + G109A + | liquor |
| F113Q + R116Q + W167F + Q172G + A174S + G182* + | |
| D183* + G184T + N195F + V206L + K391A + P473R + G476K | |
| Protease: SEQ ID NO: 24 | 0.519 mg enzyme protein/g wash |
| | liquor |
| Licheninase SEQ ID NO: 23 | 0.140 mg enzyme protein/g wash |
| | liquor |

Once the plates were washed, the performance of the wash is evaluated by a trained test person according to Assay I. The result of the evaluation is shown in table 9 below.

A trained test person has been evaluating the cleanliness of all plates relative to each other (Assay I). It is evident from the table that a small performance improvement was present when the plates where treated with licheninase compared to similar treatments without licheninase (Table 1). This indicates a performance benefit by addition of licheninase.

TABLE 9
A trained test person has been evaluating the cleanliness of all plates
relative to each other (Assay I). This table shows he cleaning results
on a scale from 1 to 10 with the higher value the cleaner.

| | Treatment | | | | | |
|---|---|---|---|---|---|---|
| | | | C | | | |
| | | B | Amylase | | | F |
| Plate | A | Amylase | Protease | D | E | Amylase |
| number | No enzymes | protease | licheninase | Licheninase | Amylase | licheninase |
| 1 | 3 | 10 | 9 | 5 | 8 | 10 |
| 2 | 1 | 8 | 9 | 4 | 10 | 10 |
| 3 | 1 | 6 | 10 | 2 | 7 | 10 |
| 4 | 3 | 10 | 9 | 2 | 5 | 7 |
| 5 | 1 | 2 | 4 | 1 | 4 | 3 |
| 6 | 2 | 7 | 9 | 1 | 6 | 5 |
| Average | 1.8 | 7.2 | 8.3 | 2.5 | 6.7 | 7.5 |
| score | | | | | | |

### Example 4

### Warewash Trials on Plates

Oat porridge soil and pre-soiled plates are prepared as described in example 1, first paragraph with the modification that after the oat porridge have boiled for 10 minutes the oat porridge is thoroughly blended for approximately 5 minutes using a hand blender.

The presoiled plates were washed during 1 cleaning cycle in a Hobart AUXX hooded warewasher at same washing program as described in example 1, second paragraph.

The cleaning performance of three different treatments were tested using (A) a conventional commercial high pH warewash detergent composition Suma Ultra Pur-Eco L2 from Sealed Air Diversey, (B) a commercial mild in-wash pH warewash detergent composition Neodisher BioClean from Dr. Weigert and (C) the Neodisher BioClean from Dr. Weigert with addition of protease and amylase. Six plates were tested for each treatment. To eliminate the risks of transferring organic materials between each setup the warewasher is purged (turned off) and restarted after each cleaning cycle. During this process the inner surface of the warewasher is cleaned.

In experiments the appropriate warewashing detergent according to the treatment was added to the warewasher at 2 g/L wash liquor. The application pH for treatment (A) was above 11, and for treatment (B) and (C) the application pH was 8.5, i.e. enzyme friendly. In the wash trials the enzymes (see Table 10) were added to the warewasher as shown.

TABLE 10
Enzymes used in the wash trials

| Enzymes | Concentration used in wash trials |
|---|---|
| Amylase: | 0.286 mg enzyme |
| SEQ ID NO: 4 + H1* + N54S + V56T + K72R + G109A + | protein/g wash liquor |
| F113Q + R116Q + W167F + Q172G + A174S + G182* + | |
| D183* + G184T + N195F + V206L + K391A + P473R + G476K | |
| Protease: SEQ ID NO: 24 | 0.519 mg enzyme |
| | protein/g wash liquor |

Once the plates were washed, the performance of the wash is evaluated by a trained test person according to Assay I. The result of the evaluation is shown in Table 11 below.

A trained test person has been evaluating the cleanliness of all plates relative to each other (Assay I). It is evident that a significant performance improvement was present when protease and amylase (Table 1) was added compared to similar treatment without protease and amylase. Surprisingly the wash performance of a mild pH (8.5) warewashing detergent in combination with protease and amylase (Table 11) was also significantly improved compared to a conventional high pH warewashing detergent.

TABLE 11
A trained test person has been evaluating the cleanliness of all plates
relative to each other (Assay I). This table shows the cleaning results
on a scale from 1 to 10 with the higher value the cleaner.

| | Treatment | | |
|---|---|---|---|
| | A | | C |
| | No enzymes | B | Amylase |
| | pH is above | No enzymes | Protease |
| Plate number | 11 | pH = 8.5 | pH = 8.5 |
| 1 | 2 | 1 | 10 |
| 2 | 1 | 1 | 10 |
| 3 | 1 | 1 | 10 |
| 4 | 1 | 1 | 10 |
| 5 | 2 | 1 | 9 |
| 6 | 1 | 1 | 9 |
| Average score | 1.3 | 1 | 9.7 |

### Example 5

### Warewash Trials on Plates

Egg yolk is prepared by separating the egg yolk of the fresh raw organic eggs. The egg yolk is stirred in a beaker, and using a brush 1 g±0.1 g is spread evenly on the inner plate surface.
The rim of the plates is kept free. Diameter of inner plate is 15 cm. The soiled plates are dried at room temperature for at least 4 hours (h) and max 24 h. For denaturation, the plates are immersed for 30 seconds in boiling demineralized water. Shortly after all the soiled plates have been boiled, they are dried for 30 minutes at 80° C. in a thermal cabinet. The plates are then stored for at least 24 h at room temperature before they are used.
The presoiled plates were washed during 1 cleaning cycle in a Hobart AUXX hooded warewasher at same washing program as described in example 1, second paragraph.
The cleaning performance of two different treatments were tested using the conventional commercial high pH warewash detergent composition Suma Ultra Pur-Eco L2 from Sealed Air Diversey (A) without enzymes and (B) with protease. Five plates were tested for each treatment. To eliminate the risks of transferring organic materials between each setup the warewasher is purged (turned off) and restarted after each cleaning cycle. During this process the inner surface of the warewasher is cleaned.
In experiments the warewashing detergent was added to the warewasher at 2 g/L wash liquor. The application pH for both treatments was 11. In the wash trials the protease (see Table 12) were added to the warewasher as shown.

TABLE 12
Enzymes used in the wash trials

| Enzymes | Concentration used in wash trials |
|---|---|
| Protease: SEQ ID NO: 24 | 0.519 mg enzyme protein/g wash |
| | liquor |

Once the plates were washed, the performance of the wash is evaluated by a trained test person according to Assay I. The result of the evaluation is shown in Table below
A trained test person has been evaluating the cleanliness of all plates relative to each other (Assay I). This figure shows the average score from results shown in Table 13 for treatment A and B. It is evident that a significant performance improvement was present when protease (12) was added compared to similar treatment without protease. Surprisingly this indicates that protease acts in a very short time interval even though pH is at 11 and that conventional warewashing detergents achieve performance improvements on this protein specific soil by adding protease.

TABLE 13
Two trained test persons have been evaluating the cleanliness
of all plates relative to each other (Assay I). This
table shows the average cleaning results on a scale from
1 to 10 with the higher value the cleaner.

Treatment

| | A | B |
|---|---|---|
| Plate number | No enzymes | Protease |
| 1 | 2.5 | 9.0 |
| 2 | 4.0 | 7.5 |
| 3 | 3.0 | 9.0 |
| 4 | 1.0 | 10.0 |
| 5 | 6.0 | 9.0 |
| 6 | 2.5 | 9.0 |
| Average score | 3.2 | 8.9 |

1. A process for removing and/or reducing soil on a surface, and/or for reducing redeposition, comprising exposing a surface to an amylase and/or protease for a time period of 10 to 240 seconds.

2. The process according to claim 1, wherein the amylase and/or protease is comprised in a liquid having a pH in the range of 7-10.5.

3. The process according to claim 1, wherein an amylase is used and the amylase has an amino acid sequence that is at least 70% identical to the amino acid sequence of SEQ ID NO: 1, 2, 3 or 4.

4. The process according to claim 3, wherein the amylase is an alpha-amylase variant comprising a modification in one or more positions corresponding to positions H1, N54, V56, K72, G109, F113, R116, T134, W140, W159, W167, Q169, Q172, L173, A174, R181, G182, 0183, G184, W189, E194, N195, V206, G255, N260, F262, A265, W284, F289, S304, G305, W347, K391, Q395, W439, W469, R444, F473, G476, and G477 of SEQ ID NO: 4, wherein said alpha-amylase variant has an amino acid sequence that is at least 75%, but less than 100%, identical to the amino acid sequence of SEQ ID NO: 4 and wherein said alpha-amylase variant has alpha-amylase activity.

5. The process according to claim 4, wherein said modification in one or more positions is selected from the group consisting of: H1*, H1A, N54S, V56T, K72R, G109A, F113Q, R116Q, R116H, T134E, W140Y, W140F, W140H, W159Y, W159F, W159H, W167Y, W167H, W167F, Q169E, Q172K, Q172G, Q172N, L173P, A174*, A174S, R181*, G182*, D183*, G184*, G184T, W189Y, W189F, W189H, W189E, W189D, W189Q, W189N, E194D, E194N, E194S, N195F, V206L, V206F, V206Y, G255A, N260G, N260P, N260A, N260G, N260P, N260A, A265G, W284G, W284H, F289H, S304K, S304R, S304Q, S304E, G305K, G305R, G305Q, G305E, W347Y, W347F, W347H, K391A, Q395P, W439N, W439Q, W439T, R444Q, W469T, W469N, F473R, G476R, G476Q, G476E, G476K G477K, G477R, G477Q, and G477E wherein the positions correspond to positions of SEQ ID NO: 4.

6. The process according to claim 5, wherein said at least one alpha-amylase variant comprises a deletion in the positions corresponding to R181+G182; R181+D183; R181+G184; G182+D183; G182+G184; or D183+G184 of SEQ ID NO:4.

7. The process according to claim 5, wherein said alpha-amylase variant is selected from the group consisting of:
H1*+N54S+V56T+G109A+Q169E+Q172K+A174*+G182*+D183*+N195F+V206L+K391A +
G476K;
H1*+N54S+V56T+G109A+R116H+A174S+G182*+D183*+N195F+V206L+K391A+G476K;
H1*+N54S+V56T+K72R+G109A+F113Q+R116Q+W167F+Q172G+A174S+G182*+D183*+
G184T+N195F+V206L+K391A+P473R+G476K;
H1*+N54S+V56T+G109A+F113Q+R116Q+Q172N+A174S+G182*+D183*+N195F+V206 L+
A265G+K391A+P473R+G476K;
H1*+N54S+V56T+K72R+G109A+F113Q+W167F+Q172R+A174S+G182*+D183*+N195F+
V206L+K391A+G476K;
H1*+N54S+V56T+K72R+G109A+R116H+T134E+W167F+Q172G+L173V+A174S+G182*+
D183*+N195F+V206L+G255A+K391A+G476K;
H1*+N54S+V56T+K72R+G109A+R116H+T134E+W167F+Q172G+L173V+A174S+G182*+
D183*+N195F+V206L+G255A+K391A+Q395P+T444Q+P473R+G476K;
H1*+N54S+V56T+G109A+T134E+A174S+G182*+D183*+N195F+V206L+K391A+G476K;
H1*+N54S+V56T+K72R+G109A+A174S+G182*+D183*+N195F+V206L+G255A+K391A+G476K;
H1*+N54S+V56T+K72R+G109A+F113Q+R11Q+R+W167F+Q172G+A174S+G184T+N, 195
F+V206L+K391A+P473R+G476K, and
H1*+N54S+V56T+G109A+W167F+Q172E+L173P+A174K+G182*+D183*+N, 195F+V206L+K391A+G476K, of the polypeptide of SEQ ID NO: 4, and wherein said alpha-amylase variant has an amino acid sequence that is at least 80%, but less than 100% identical to the amino acid sequence of SEQ ID NO: 4 and wherein said alpha-amylase variant has alpha-amylase activity.

8. The process according to claim 1, wherein a protease is used, and the protease is selected from the group consisting of
i. a protease having an amino acid sequence that is at least 70% identical to the amino acid sequence of SEQ ID NOs: 5, 6, 7 or 8; and
ii. a protease variant comprising a substitution at one or more positions corresponding to positions 9, 15, 36, 61, 68, 76, 99, 106, 120, 167, 170, 194, 195, 205, 218, 235, 245 or 261 of SEQ ID NO: 3, wherein said protease variant has an amino acid sequence that is at least 75%, but less than 100%, identical to the amino acid sequence of SEQ ID NO: 5,
iii. the protease of SEQ ID NO: 24.

9. The process according to claim 8, wherein the protease is selected from the group consisting of: M222S, *36D+N76D+N120D+G195E+K235L, Y167A+R170S+A194P, S99SE, V68A+S106A, S9R+A15T+V68A+N218D+Q245R, S9R+A15T+G61E+V68A+A194P+V205I+Q245R+N261D and S99AD of SEQ ID NO: 5, wherein said protease variant has an amino acid sequence that is at least 75%, but less than 100%, identical to the amino acid sequence of SEQ ID NO: 5, and wherein said protease variant has protease activity.

10. The process according to claim 1, further comprising exposing the surface to an additional enzyme selected from the group consisting of a protease, lipase, cutinase, an amylase, carbohydrase, cellulase, pectinase, mannanase, arabinase, galactanase, xylanase, oxidase, a licheninase, a laccase, a peroxidase, and combinations thereof.

11. The process according to claim 1, wherein the surface is the inner surface of a warewashing machine or the surface of a ware.

12. A warewashing detergent composition comprising an amylase and/or a protease and one or more detergent components which composition has a pH in the range of 7-10.5.

13. The composition according to claim 12, wherein the detergent component is selected from the group consisting of as surfactants, builders, chelators or chelating agents, bleach system or bleach components, polymers, foam boosters, suds suppressors, dyes, perfume, tannish inhibitors, optical brighteners, bactericides, fungicides, soil suspending agents, anti-corrosion agents, enzyme stabilizers, enzyme inhibitors or activators, transferase(s), hydrolytic enzymes, oxido reductases, bluing agents and fluorescent dyes, antioxidant, and solubilizers.

14. The composition according to claim 12, wherein the composition further comprises one or more protease inhibitors of which at least one is a peptide aldehyde, a hydrosulfite adduct or a hemiacetal adduct thereof.

15. The composition according to claim 12, wherein the composition comprises an alpha-amylase.

16. The composition according to claim 15, wherein alpha-amylase has an amino acid sequence that is at least 70% identical to the sequence of SEQ ID NO: 1, 2, 3 or 4.

17. The composition according to claim 16, wherein the amylase is an alpha-amylase variant comprising a modification in one or more positions corresponding to positions H1, N54, V56, K72, G109, F113, R116, T134, W140, W159, W167, Q169, Q172, L173, A174, R181, G182, D183, G184, W189, E194, N195, V206, G255, N260, F262, A265, W284, F289, S304, G305, W347, K391, Q395, W439, W469, R444, F473, G476, and G477 of SEQ ID NO: 4, wherein said alpha-amylase variant has an amino acid sequence that is at least 75%, but less than 100%, identical to the amino acid sequence of SEQ ID NO: 4 and wherein said alpha-amylase variant has alpha-amylase activity.

18. The composition according to claim 16, wherein said modification in one or more positions is selected from the group consisting of: H1*, H1A, N54S, V56T, K72R, G109A, F113Q, R116Q, R116H, T134E, W140Y, W140F, W140H, W159Y, W159F, W159H, W167Y, W167H, W167F, Q169E, Q172K, Q172G, Q172N, L173P, A174*, A174S, R181*, G182*, D183*, G184*, G184T, W189Y, W189F, W189H, W189E, W189D, W189Q, W189N, E194D, E194N, E194S, N195F, V206L, V206F, V206Y, G255A, N260G, N260P, N260A, N260G, N260P, N260A, A265G, W284G, W284H, F289H, S304K, S304R, S304Q, S304E, G305K, G305R, G305Q, G305E, W347Y, W347F, W347H, K391A, Q395P, W439N, W439Q, W439T, R444Q, W469T, W469N, F473R, G476R, G476Q, G476E, G476K G477K, G477R, G477Q, and G477E wherein the positions correspond to positions of SEQ ID NO: 4.

19. The composition according to claim 16, wherein said at least one alpha-amylase variant comprises a deletion in the positions corresponding to R181+G182; R181+D183; R181+G184; G182+D183; G182+G184; or D183+G184 of SEQ ID NO:4.

20. The composition according to claim 16, wherein said alpha-amylase variant is selected from the group consisting of:
H1*+N54S+V56T+G109A+Q169E+Q172K+A174*+G182*+D183*+N, 195F+V206L+K391A+G476K;
H1*+N54S+V56T+G109A+R116H+A174S+G182*+D183*+N, 195F+V206L+K391A+G476K;
H1*+N54S+V56T+K72R+G109A+F113Q+R116Q+W167F+Q172G+A174S+G182*+D183*+
G184T+N, 195F+V206L+K391A+P473R+G476K;
H1*+N54S+V56T+G109A+F113Q+R116Q+Q172N+A174S+G182*+D183*+N, 195F+V206 L+
A265G+K391A+P473R+G476K;
H1*+N54S+V56T+K72R+G109A+F, 113Q+W167F+Q172R+A174S+G182*+D183*+N, 195F+V206L+K391A+G476K;
H1*+N54S+V56T+K72R+G109A+R116H+T134E+W167F+Q172G+L173V+A174S+G182*+D183*+N, 195F+V206L+G255A+K391A+G476K;
H1*+N54S+V56T+K72R+G109A+R116H+T134E+W167F+Q172G+L173V+A174S+G182*+D183*+N, 195F+V206L+G255A+K391A+Q395P+T444Q+P473R+G476K;
H1*+N54S+V56T+G109A+T134E+A174S+G182*+D183*+N, 195F+V206L+K391A+G476K;
H1*+N54S+V56T+K72R+G109A+A174S+G182*+D183*+N, 195F+V206L+G255A+K391A+G476K;
H1*+N54S+V56T+K72R+G109A+F113Q+R116Q+W167F+Q172G+A174S+G184T+N, 195 F+V206L+K391A+P473R+G476K, and
H1*+N54S+V56T+G109A+W167F+Q172E+L173P+A174K+G182*+D183*+N, 195F+V206L+K391A+G476K, of the polypeptide of SEQ ID NO: 4 and wherein said alpha-amylase variant shares at least 80%, such as at least 85%, such as at least 90%, such as at least 93%, such as at least 94%, such as at least 95%, such as at least 96%, such as at least 97%, such as at least 98%, but less than 100% sequence identity with the polypeptide of SEQ ID NO: 4 and wherein said alpha-amylase variant has alpha-amylase activity.

21. The composition according to claim 20, wherein the composition comprises a protease, and the protease is selected from the group consisting of
i. a protease having an amino acid sequence that is at least 70% identical to the amino acid sequence of SEQ ID NOs: 5, 6, 7 or 8;
ii. a protease variant comprising a substitution at one or more positions corresponding to positions 9, 15, 36, 61, 68, 76, 99, 106, 120, 167, 170, 194, 195, 205, 218, 235, 245 or 261 of SEQ ID NO: 3, wherein said protease variant has an amino acid sequence that is at least 75%, but less than 100%, identical to the amino acid sequence of SEQ ID NO: 5; and
iii. the protease of SEQ ID NO: 24.

22. The composition according to claim 21, wherein the protease variant is selected from the group consisting of: M222S, *36D+N76D+N120D+G195E+K235L, Y167A+R170S+A194P, S99SE, V68A+S106A, S9R+A15T+V68A+N218D+Q245R, S9R+A15T+G61E+V68A+A194P+V205I+Q245R+N261D and S99AD of SEQ ID NO: 5, wherein said protease variant has an amino acid sequence that is at least 75%, but less than 100%, identical to the amino acid sequence of SEQ ID NO: 5 and wherein said protease variant has protease activity.

23. The composition according to claim 12, further comprises an additional enzyme selected from the group consisting of a protease, lipase, cutinase, amylase, carbohydrase, cellulase, pectinase, mannanase, arabinase, galactanase, xylanase, oxidase, licheninase, laccase, and peroxidase, and combinations thereof.

24. A method for removing and/or reducing soil on a surface, wherein a cleaning cycle comprises the steps of:
i. a washing step, wherein the surface is exposed to a wash liquor comprising
a. an amylase and/or a protease and optionally detergent components, or
b. a warewashing detergent composition according to claim 12,
ii. optionally draining part of the wash liquor,
iii. optionally rinsing the surface,
iv. optionally drying the surface;
wherein the surface is exposed to the wash liquor for a time period in the range of 10 to 240 seconds.


SEQUENCE LISTING (24 sequences)

SEQ ID NO: 1 - protein, 485 aa, Bacillus sp.
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 2 - protein, 485 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 3 - protein, 483 aa, Bacillus licheniformis [sequence omitted]

SEQ ID NO: 4 - protein, 481 aa, Artificial sequence
  Fusion protein
VNGTLMQYFEWYTPNDGQHWKRLQNDAEHLSDIGITAVWIPPAYKGTSQADVGYGAYDLYDLGEFHQKGTVRTKYGTKGELQSAIKSLHSRDINVYGDVVINHKGGADATEDVTAVEVDPADRNRVISGEHLIKAWTHFHFPGRGSTYSDFKWHWYHFDGTDWDESRKLNRIYKFQGKAWDWEVSNENGNYDYLMYADIDYDHPDVAAEIKRWGTWYANELQLDGFRLDAVKHIKFSFLRDWVNHVREKTGKEMFTVAEYWQNDLGALENYLNKTNFNHSVFDVPLHYQFHAASTQGGGYDMRKLLNGTVVSKHPLKSVTFVDNHDTQPGQSLESTVQTWFKPLAYAFILTRESGYPQVFYGDMYGTKGDSQREIPALKHKIEPILKARKQYAYGAQHDYFDHHDIVGWTREGDSSVANSGLAALITDGPGGAKRMYVGRQNAGETWHDITGNRSEPVVINSEGWGEFHVNGGSVSIYVQR

SEQ ID NO: 5 - protein, 269 aa, Bacillus lentus
AQSVPWGISRVQAPAAHNRGLTGSGVKVAVLDTGISTHPDLNIRGGASFVPGEPSTQDGNGHGTHVAGTIAALNNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNVQSTYPGSTYASLNGTSMATPHVAGAAALVKQKNPSWSNVQIRNHLKNTATSLGSTNLYGSGLVNAEAATR

SEQ ID NO: 6 - protein, 274 aa, Bacillus licehniformis
AQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYARGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 7 - protein, 268 aa, Bacillus halodurans
QTVPWGISFINTQQAHNRGIFGNGARVAVLDTGIASHPDLRIAGGASFISSEPSYHDNNGHGTHVAGTIAALNNSIGVLGVAPSADLYAVKVLDRNGSGSLASVAQGIEWAINNNMHIINMSLGSTSGSSTLELAVNRANNAGILLVGAAGNTGRQGVNYPARYSGVMAVAAVDQNGQRASFSTYGPEIEISAPGVNVNSTYTGNRYVSLSGTSMATPHVAGVAALVKSRYPSYTNNQIRQRINQTATYLGSPSLYGNGLVHAGRATQ

SEQ ID NO: 8 - protein, 275 aa, Bacillus amyloliquefaciens [sequence omitted]

SEQ ID NO: 9 - protein, 4 aa, Artificial
  Subtilisin inhibitor
  MISC_FEATURE (1)..(1): Acetyl-Phe
  MISC_FEATURE (4)..(4): Tyr-H
FGAY

SEQ ID NO: 10 - protein, 4 aa, Artificial
  Subtilisin inhibitor
  MISC_FEATURE (1)..(1): Acetyl-Leu
  MISC_FEATURE (4)..(4): Tyr-H
LGAY

SEQ ID NO: 11 - protein, 4 aa, Artificial
  Subtilisin inhibitor
  MISC_FEATURE (1)..(1): Acetyl-Tyr
  MISC_FEATURE (4)..(4): Tyr-H
YGAY

SEQ ID NO: 12 - protein, 4 aa, Artificial
  Subtilisin inhibitor
  MISC_FEATURE (1)..(1): Acetyl-Phe; MeO-CO-Phe; MeSO2-Phe; or EtSO2-Phe
  MISC_FEATURE (4)..(4): Leu-H
FGAL

SEQ ID NO: 13 - protein, 4 aa, Artificial
  Subtilisin inhibitor
  MISC_FEATURE (1)..(1): Acetyl-Phe or MeO-CO-Phe
  MISC_FEATURE (4)..(4): Tyr-H
FGAF

SEQ ID NO: 14 - protein, 4 aa, Artificial
  Subtilisin inhibitor
  MISC_FEATURE (1)..(1): Acetyl-Phe
  MISC_FEATURE (4)..(4): Tyr-H
FGVY

SEQ ID NO: 15 - protein, 4 aa, Artificial
  Subtilisin inhibitor
  MISC_FEATURE (1)..(1): Acetyl-Phe
  MISC_FEATURE (4)..(4): Met-H
FGAM

SEQ ID NO: 16 - protein, 4 aa, Artificial
  Subtilisin inhibitor
  MISC_FEATURE (1)..(1): Acetyl-Trp
  MISC_FEATURE (4)..(4): Tyr-H
WLVY

SEQ ID NO: 17 - protein, 4 aa, Artificial
  Subtilisin inhibitor
  MISC_FEATURE (1)..(1): MeO-P(OH)(O)-Leu
  MISC_FEATURE (4)..(4): Leu-H
LGAL

SEQ ID NO: 18 - protein, 237 aa, Bacillus agaradhaerens
MLTLLMMSFAGAAYAHNPVTDEEVYHSFNSHDWQNWNMSDGWKNDDYFFGCHWSQNRVNFYGGQMELSLRTNYSYAPPYNYECAEYTTNNFYGYGLYEVSMKPAKVSGVISSFFTYTGPSYNGAPWDEIDIEFLGNDTTKVQFNYYTDGVGGNEILYDLGFDAADSYNTYAFDWQENYINWYVNGQLVATATENIPSNPSKIMMNIWNTYGIDEWAGRYYGEDANASYNWVRYTPNR

SEQ ID NO: 19 - protein, 379 aa, Bacillus sp-62449
VVKIKINNSIRIVMLTLIMMSVSVVAYAYNPVTEDELYHSFDSHDARNWQISDGWRNGDDFFGCHWSQNRVNFNRGEMELSLRTNYSYSAPYNYECAEYATSNFYGYGLYEVSMKPANVSGVISSFFTYTGPSYNGAPWDEIDIEFLGNDTTKVQFNYYTNGVGGNEIIYDLGFDAANSFNTYAFDWQENYISWYVNGNLVATATENIPSNPSKIMMNVWNTYGIDEWAGAYGGEAANATYEWVRYTPNNGNTTPSTAPDFQLQACDYSDSSGITSWSCGVGTFHSSNWIKFDSVDLSTGYNAFAVSYTSPGSGSFDIRLGSPHGQRIGTVNYGATGGWSNYEWSGTPSLDVTVRGAHDIYIVATSGAANLREFWFKNE

SEQ ID NO: 20 - protein, 379 aa, Bacillus sp-62449
MVKIKINNSIRIVMLTLIMMSVSVVAYAYNPVTEDELYHSFDSHDARNWQISDGWRNGDDFFGCHWSQNRVNFNRGEMELSLRTNYSYSAPYNYECAEYATSNFYGYGLYEVSMKPANVSGVISSFFTYTGPSYNGAPWDEIDIEFLGNDTTKVQFNYYTNGVGGNEIIYDLGFDAANSFNTYAFDWQENYISWYVNGNLVATATENIPSNPSKIMMNVWNTYGIDEWAGAYGGEAANATYEWVRYTPNNGNTTPSTAPDFQLQACDYSDSSGITSWSCGVGTFHSSNWIKFDSVDLSTGYNAFAVSYTSPGSGSFDIRLGSPHGQRIGTVNYGATGGWSNYEWSGTPSLDVTVRGAHDIYIVATSGAANLREFWFKNE

SEQ ID NO: 21 - protein, 276 aa, Bacillus akibai
MKKKFVLFSMCLLLFSGLITGLVQSPQVAEAAERPIGTTFVETFESYDSERWSKAGVWTNGQMFNATWYPEQVTFSDGKMKLQIDKEDNETASPPYKAGELRTNDFYHYGLFEVSMKPAKSTGTVSSFFTYTGPWDWDNDPWDEIDIEFLGKDTTKIQFNYFTNGVGGNEHYHELGFDAADDFNTYAFEWRPESIRWFVNGELVHTATENIPQTPQKIMMNLWPGIGVDGWTGRFNGEDTPVVTQYDWVKYTPLEELGCYNEKNNKYKKCKKTKVK

SEQ ID NO: 22 - protein, 243 aa, Bacillus mojavensis
MSYRMKRVLLLLVTGLFMSLSAFTSTASAQTGGSFFDPFNGYNSGFWQKANGYSNGNMFNCTWRANNVSMTSLGEMRLALTSPSYNKFDCGENRSVQTYGYGLYEVRMKPAKNVGIVSSFFTYTGPTDGTPWDEIDIEFLGKDTTKVQFNYYTNGVGNHEKLVDLGFDAANAYHTYAFDWQPNSIKWYVDGQLKHTATSQIPTTPGKIMMNLWNGTGVDEWLGSYNGVTPLYAHYDWVRYTKK

SEQ ID NO: 23 - protein, 230 aa, Artificial Sequence
  His-tagged mature protein (Bacillus agaradhaerens)
HHHHHHPRHNPVTDEEVYHSFNSHDWQNWNMSDGWKNDDYFFGCHWSQNRVNFYGGQMELSLRTNYSYAPPYNYECAEYTTNNFYGYGLYEVSMKPAKVSGVISSFFTYTGPSYNGAPWDEIDIEFLGNDTTKVQFNYYTDGVGGNEILYDLGFDAADSYNTYAFDWQENYINWYVNGQLVATATENIPSNPSKIMMNIWNTYGIDEWAGRYYGEDANASYNWVRYTPNR

SEQ ID NO: 24 - protein, 269 aa, Artificial Sequence
  Variant of Savinase
AQSVPWGIERVQAPAAHNRGLTGSGVKVAVLDTGISTHPDLRIRGGASFVPGEPSTQDGNGHGTHVAGTIAALDNSIGVLGVAPSAELYAVKVLGASGSGSVSSIAQGLEWAGNNGMHVANLSLGSPSPSATLEQAVNSATSRGVLVVAASGNSGAGSISYPARYANAMAVGATDQNNNRASFSQYGAGLDIVAPGVNILSTWPGSTYASLNGTSMATPHVAGAAALVKQKNPSWSNVQIRNHLKNTATSLGDTWEYGSGLVNAEAATR